SEQUENCE LISTING (27 sequences)

SEQ ID NO: 1 - protein, 107 aa, unidentified
DIQLTQSPLSLSASAGDRVTITCRASQSIRDYLGWYQQKPGKAPKLLIYAASKLQSGVPSRFSGSGSGTDFTLTISSLQPEDFATYYCQQSYHTPPFTFGQGTKVEI

SEQ ID NO: 2 - protein, 122 aa, unidentified
EVQLLESGGGLVQPGGSLRLTCAASGFTFSSYAMSWVRQAPGKGLEWVSVINGRGSNTYYADSVKGRFTITRDNSKNTLYLEMNSLRAEDTAVYYCARVRAYSGPSYGFDYWGQGTLVTVSS

SEQ ID NO: 3 - protein, 6 aa, unidentified
QSIRDY

SEQ ID NO: 4 - skipped

SEQ ID NO: 5 - protein, 10 aa, unidentified
QQSYHTPPFT

SEQ ID NO: 6 - protein, 8 aa, unidentified
GFTFSSYA

SEQ ID NO: 7 - protein, 7 aa, unidentified
NGRGSNT

SEQ ID NO: 8 - protein, 15 aa, unidentified
ARVRAYSGPSYGFDY

SEQ ID NO: 9 - protein, 237 aa, Homo sapiens
MDMRVPAQLLGLLLLWLSGARCDIQLTQSPLSLSASAGDRVTITCRASQSIRDYLGWYQQKPGKAPKLLIYAASKLQSGVPSRFSGSGSGTDFTLTISSLQPEDFATYYCQQSYHTPPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 215 aa, Homo sapiens
DIQLTQSPLSLSASAGDRVTITCRASQSIRDYLGWYQQKPGKAPKLLIYAASKLQSGVPSRFSGSGSGTDFTLTISSLQPEDFATYYCQQSYHTPPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 213 aa, Homo sapiens
DIQLTQSPLSLSASAGDRVTITCRASQSIRDYLGWYQQKPGKAPKLLIYAASKLQSGVPSRFSGSGSGTDFTLTISSLQPEDFATYYCQQSYHTPPFTFGQGTKVEIKGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 12 - protein, 471 aa, Homo sapiens
MELGLSWIFLLAILKGVQCEVQLLESGGGLVQPGGSLRLTCAASGFTFSSYAMSWVRQAPGKGLEWVSVINGRGSNTYYADSVKGRFTITRDNSKNTLYLEMNSLRAEDTAVYYCARVRAYSGPSYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVTKSEWQQGFVFSCSVMHEALHNHYTQKSLSLSPGK

SEQ ID NO: 13 - protein, 452 aa, Homo sapiens
EVQLLESGGGLVQPGGSLRLTCAASGFTFSSYAMSWVRQAPGKGLEWVSVINGRGSNTYYADSVKGRFTITRDNSKNTLYLEMNSLRAEDTAVYYCARVRAYSGPSYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESYGTEWSSYKTTPPVLDSDGSFFLYSKLTVTKSEWQQGFVFSCSVMHEALHNHYTQKSLSLSPGK

SEQ ID NO: 14 - protein, 471 aa, Homo sapiens
MELGLSWIFLLAILKGVQCEVQLLESGGGLVQPGGSLRLTCAASGFTFSSYAMSWVRQAPGKGLEWVSVINGRGSNTYYADSVKGRFTITRDNSKNTLYLEMNSLRAEDTAVYYCARVRAYSGPSYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 452 aa, Homo sapiens
EVQLLESGGGLVQPGGSLRLTCAASGFTFSSYAMSWVRQAPGKGLEWVSVINGRGSNTYYADSVKGRFTITRDNSKNTLYLEMNSLRAEDTAVYYCARVRAYSGPSYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 9 aa, unidentified
DAGDLWFPG

SEQ ID NO: 17 - protein, 220 aa, unidentified
AIRMTQSPDSLPVSLGERATINCKSSQSVLYGSNNKNYLAWYQQKPGQPPKLLIYWASTRESGVPDRFSGSGSGTDFTLTISSLQAEDVAVYYCQQYYSTPLTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 447 aa, unidentified
EVQLVQSGGGLVQPGGSLRLSCAASGFTFSSYAMSWVRQAPGKGLEWVSAISGSGGSTYYADSVKGRFTISRDNAKRSLYLQMNDLRVEDTAVYYCARTRTNVFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 217 aa, unidentified
DIRLTQPPSVSGAPGQRVTISCSGSSSNIGSLFVSWYQQLPGTAPKLLIYSNSQHPSGVPDRFSGSKSGTSASLAITGLQAEDEADYYCSAYDQFSNSVVFGGGTKLTVLGQVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 215 aa, unidentified
DIRLTQPPSVSGAPGQRVTISCSGSSSNIGSLFVSWYQQLPGTAPKLLIYSNSQHPSGVPDRFSGSKSGTSASLAITGLQAEDEADYYCSAYDQFSNSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 21 - protein, 453 aa, unidentified
EVQLVQSGGGLVQPGGSLRLSCAASGFTFSSYGITWVRQAPGKGLEWVSFISGGGSYTYYADSVKGRFTISRDNAKRTLYLQMNSLRAEDTAVYYCARSGRAYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKYTEWSS

SEQ ID NO: 22 - protein, 217 aa, unidentified
DIVMTQPPSVSVTPGQRVTISCRSSSSNIGSLFVSWYQQLPGTAPKLLIYSNSQHPSGVPDRFSGSKSGTSASLAITGLQAEDEADYYCSAYDQFSNSVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 23 - protein, 215 aa, unidentified
DIVMTQPPSVSVTPGQRVTISCRSSSSNIGSLFVSWYQQLPGTAPKLLIYSNSQHPSGVPDRFSGSKSGTSASLAITGLQAEDEADYYCSAYDQFSNSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEA

SEQ ID NO: 24 - protein, 453 aa, unidentified
EVQLLESGGGLVQPGGSLRLSCAASGFTFSSNAMTWVRQAPGKGLEWVSVIGSSGSYTYYADSVKGRFTISRDNSKNTLYLQMNSLRAEDTAVYYCARSGTTGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKYTEWSS

SEQ ID NO: 25 - DNA, 723 bp, Homo sapiens
  source 1..723: mol_type = other DNA
catcatggacatgagagtgcccgctcagctgctgggactgctgttgttgtggctgtctggcgctagatgcgacatccagctgacccagtctccactgtctctgtctgcctctgctggcgacagagtgaccatcacctgtcgggcctctcagtctatcagagactacctcggctggtatcagcagaagcctggcaaggctcccaagctgctgatctacgctgcctctaaactgcagtccggcgtgccctctagattctctggctctggatctggcaccgacttcaccctgaccatcagttctctgcagcctgaggacttcgccacctactactgccagcagtcctatcacacccctccattcacctttggccagggcaccaaggtggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtctggcaactcccaagagtctgtgaccgagcaggactccaaggactctacctacagcctgtcctccacactgaccctgtctaaggccgactacgagaagcacaaggtgtacgcctgtgaagtgacccaccagggactgtctagccccgtgaccaagtctttcaacagaggcgagtgctgattaat

SEQ ID NO: 26 - DNA, 1424 bp, unidentified
  source 1..1424: mol_type = other DNA
ctaggatggaactgggcctgtcctggatctttctgctggctattctgaagggcgtgcagtgcgaagtgcagctgttggaatctggcggaggattggttcagcctggcggctctctgagactgacctgtgctgcctctggcttcaccttctcctcttacgccatgtcctgggtccgacaggctcctggaaaaggactggaatgggtgtccgtgatcaacggcagaggctccaacacctactacgccgactctgtgaagggcagattcaccatcaccagagacaactccaagaacaccctgtacctggaaatgaactccctgagagccgaggacaccgccgtgtactactgtgctagagtgcgggcttactctggcccttcctacggctttgattattggggccagggcaccctggtcaccgtgtcctctgcttctacaaagggcccctctgtgttccctctggctcctagctctaagtccacctctggtggaaccgctgctctgggctgtctggtcaaggattacttccctgagcctgtgaccgtgtcttggaattccggtgctctgacctccggcgtgcacacatttccagctgtgctgcagtcctccggcctgtactctctgtcctctgtcgtgaccgtgccttctagctctctgggcacccagacctacatctgcaacgtgaaccacaagccttccaacaccaaagtggacaagaaggtggaacccaagtcctgcgacaagacccacacctgtccaccttgtcctgctccagaagctgctggcggaccctccgttttcctgtttccacctaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtctgtgctgacagtgctgcaccaggactggctgaacggcaaagagtacaagtgcaaggtgtccaacaaggccctgcctgctcctatcgaaaagaccatctccaaggctaagggccagcctcgggaacctcaggtttacacactgcctccatctcgggacgagctgaccaagaatcaggtgtccctgacctgcctcgtgaagggcttctacccttccgacattgccgtggaatgggagagctatggcaccgagtggtccagctacaagacaacccctcctgtgctggactccgacggctcattcttcctgtactccaagctgaccgtgaccaagtctgagtggcagcagggcttcgtgttctcctgctctgtgatgcacgaggccctgcacaaccactacacccagaagtccctgtctctgtcccctggcaaatgagta

SEQ ID NO: 27 - protein, 210 aa, Homo sapiens
HNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSRSLLEGEIPFPPTSILLLLACIFLIKILAASALWAAAWHGQKPGTHPPSELDCGHDPGYQLQTLPGLRDT

(19) United States
(12) Patent Application Publication
(10) Pub. No.: US 2026/0226160 A1
(43) Pub. Date: Aug. 6, 2026

(54) HUMAN ANTI-TREM2 ANTIBODY FOR TREATING NEURODEGENERATIVE DISORDERS

(71) Applicant: ISAR Bioscience GmbH, Planegg (DE)

(72) Inventors: Miriam CHRISTLMEIER, Planegg (DE); Markus KRALLER, Planegg (DE); Hans-Peter HOLTHOFF, Planegg (DE); Martin UNGERER, Planegg (DE); Katrin SIMMNACHER, Planegg (DE)

(73) Assignee: ISAR Bioscience GmbH, Planegg (DE)

(21) Appl. No.: 19/150,326

(22) PCT Filed: Jan. 29, 2024
(86) PCT No.: PCT/EP2024/052088

§ 371 Date: Jul. 23, 2025

(30) Foreign Application Priority Data
Jan. 30, 2023 (EP) 23154034.5

(51) Int. Cl. C07K 16/28; A61P 25/28

(52) U.S. Cl. CPC C07K 16/2803; A61P 25/28; C07K 2317/34; C07K 2317/75; C07K 2317/92

(57) ABSTRACT

The invention provides a protein or antibody capable of binding to human TREM2, comprising an Ig light chain variable region and an Ig heavy chain variable region, wherein the amino acid sequence of the light chain variable region is that of SEQ ID NO: 1 or is an amino acid sequence having 1 or 2 amino acid residue substitutions compared to SEQ ID NO: 1, and the amino acid sequence of the heavy chain variable region is that of SEQ ID NO: 2 or is an amino acid sequence having 1 or 2 amino acid residue substitutions compared to SEQ ID NO: 2.

### FIELD OF THE PRESENT INVENTION

The present invention relates to a protein comprising or consisting of an immunoglobulin (Ig) heavy variable region or a light chain variable region. The invention also relates to a protein comprising an antibody light chain variable region and an antibody heavy chain variable region, and to an antibody comprising an antibody light chain variable region and an antibody heavy chain variable region. The invention also relates to a pharmaceutical composition comprising the protein or antibody. The protein or antibody is capable of binding to human TREM2, preferably to the stalk region of hTREM2. The protein or antibody is generally an agonist of hTREM2, preferably an agonistic antibody against hTREM2. Accordingly, the protein, antibody and pharmaceutical composition can be used in therapy, notably for therapy or prevention of a neurodegenerative disorder, such as Alzheimer's disease. The invention also relates to a method of treating or preventing a neurodegenerative disease, such as Alzheimer's disease.

### BACKGROUND OF THE INVENTION

Neurodegenerative disorders such as Alzheimer's disease (AD) result in age-associated progressive deterioration of neuronal structures, ultimately leading to cognitive disability and dementia. AD is the most common form of dementia and affects millions of people worldwide. To date, there are only two approved antibody-based therapeutics for the treatment of AD. Aducanumab (marketed as Aduhelm) is an amyloid β (Aβ)-directed antibody and its use and approval are hotly debated. The antibody targets amyloid plaques, a key sign of Alzheimer's disease, resulting in reduced plaque load in the brain. Lecanemab (marketed as Leqembi), is another antibody just recently approved by the F.D.A. Leqembi is also directed against Aβ—more specifically against the protofibrils. Both therapeutics have a high risk of infusion-related reactions, brain edema and microhemorrhages (van Dyck et al., 2022). Out of numerous Aβ-based therapeutic approaches, Aduhelm and Leqembi are the only affirmed candidates for treatment of AD so far. Therefore, new strategies to interfere with plaque deposition are needed.

Triggering receptor expressed on myeloid cells 2 (TREM2) is a transmembrane receptor expressed on myeloid cells and is essential for activation of microglia cells. TREM2 mutations have been identified in neurodegenerative disorders, such as AD, wherein the mutations result in loss of TREM2 function through a variety of different mechanisms (Gernot Kleinberger et al., 2017; Schlepckow et al., 2017; Song et al., 2017; Ulland et al., 2017). TREM2-mediated signaling in microglia cells induces a transition of homeostatic microglia into disease-associated microglia (DAM) (Keren-Shaul et al., 2017). This transition is phenotypically characterized by enhanced phagocytosis, migration and cell survival. Activation of TREM2 signaling is mediated through the adaptor protein DAP12. Upon ligand binding to TREM2, the ITAM motif of DAP12 becomes phosphorylated, which results in the recruitment of phospho-spleen tyrosine kinase (pSYK) and activation of downstream signaling molecules. The signaling is terminated by shedding of the extracellular domain of TREM2 mediated by α-secretases, which results in the release of soluble TREM2 (sTREM2) (G Kleinberger et al., 2014; Wunderlich et al., 2013). The function of sTREM2 is not entirely clear. Altered levels of sTREM2 have been reported in the CSF of AD patients and usage as a potential disease biomarker has been suggested (Zhong & Chen, 2019). sTREM2 levels change dynamically during AD progression, with highest levels at the early symptomatic stages of the disease (Suárez-Calvet et al., 2016). Moreover, a positive correlation of STREM2 with phospho-tau and total tau can be observed (Suárez-Calvet et al., 2016). In addition, studies suggest a signaling function for sTREM2 (Zhong et al., 2017, 2019).

Departing from the prior art, it is a problem of the invention to provide a remedy for the treatment or prevention of neurodegenerative diseases/disorders, such as AD.

### SUMMARY OF THE INVENTION

In order to solve this problem, the invention provides:

1) A protein comprising or consisting of an heavy chain variable region or comprising or consisting of a light chain variable region, wherein
the amino acid sequence of the heavy chain variable region is that of SEQ ID NO: 2 or is an amino acid sequence having 1 or 2 amino acid residue substitutions compared to SEQ ID NO: 2, and
the amino acid sequence of the light chain variable region is that of SEQ ID NO: 1 or is an amino acid sequence having 1 or 2 amino acid residue substitutions compared to SEQ ID NO: 1; preferably the protein is an agonist of human TREM2.

2) The protein according to 1), comprising an antibody heavy chain comprising said heavy chain variable region.

3) The protein according to 1) or 2), comprising two identical antibody heavy chains, each heavy chain comprising said heavy chain variable region and at least one, preferably at least two Ig heavy chain constant regions.

4) A protein, preferably according to any one of 1) to 3), comprising a light chain variable region and a heavy chain variable region, wherein
the amino acid sequence of the light chain variable region is that of SEQ ID NO: 1 or is an amino acid sequence having 1 or 2 amino acid residue substitutions compared to SEQ ID NO: 1, and
the amino acid sequence of the heavy chain variable region is that of SEQ ID NO: 2 or is an amino acid sequence having 1 or 2 amino acid residue substitutions compared to SEQ ID NO: 2.

5) A protein, preferably according to 4), comprising an antibody light chain and an antibody heavy chain, wherein
the light chain comprises a light chain variable region, the amino acid sequence of said light chain variable region being that of SEQ ID NO: 1 or being an amino acid sequence having 1 or 2 amino acid residue substitutions compared to SEQ ID NO: 1, and
the heavy chain comprises a heavy chain variable region, the amino acid sequence of the heavy chain variable region being that of SEQ ID NO: 2 or being an amino acid sequence having 1 or 2 amino acid residue substitutions compared to SEQ ID NO: 2.

6) A protein, preferably according to 4) or 5), comprising a light chain variable region and a heavy chain variable region, wherein
the light chain (LC) variable region comprises, preferably in CDR-L3, a segment that has the amino acid sequence of SEQ ID NO: 5 or that has an amino acid sequence having 1 amino acid residue substitution compared to the amino acid sequence of SEQ ID NO: 5; and
the heavy chain (HC) variable region comprises, preferably in CDR-H3, a segment that has the amino acid sequence of SEQ ID NO: 8 or that has an amino acid sequence having 1 amino acid residue substitution compared to the amino acid sequence of SEQ ID NO: 8.

7) The protein according to any one of 4) to 6), wherein
the light chain variable region comprises, preferably in CDRs L1 and L3, the following amino acid segments in N-terminal to C-terminal direction:
a segment (CDR-L1) having the amino acid sequence of SEQ ID NO: 3, and
a segment (CDR-L3) having the amino acid sequence of SEQ ID NO: 5 or an amino acid sequence having 1 amino acid residue substitution compared to the amino acid sequence of SEQ ID NO: 5; and
the heavy chain (HC) variable region comprises, preferably in CDRs H2 and H3, the following amino acid segments in N-terminal to C-terminal direction:
a segment (CDR-H2) having the amino acid sequence of SEQ ID NO: 7, and
a segment (CDR-H3) having the amino acid sequence of SEQ ID NO: 8 or of an amino acid sequence having 1 amino acid residue substitution compared to the amino acid sequence of SEQ ID NO: 8.

8) The protein according to 6) or 7), wherein the light chain (LC) variable region comprises, preferably in CDRs L1 to L3, the following amino acid segments in N-terminal to C-terminal direction:
a segment (CDR-L1) having the amino acid sequence of SEQ ID NO: 3,
a segment (CDR-L2) having the amino acid sequence of SEQ ID NO: 4, and
a segment (CDR-L3) having the amino acid sequence of SEQ ID NO: 5 or having an amino acid sequence having 1 amino acid residue substitution compared to the amino acid sequence of SEQ ID NO: 5; and
the heavy chain (HC) variable region comprises, preferably in CDRs H1 to H3, the following amino acid sequence segments in N-terminal to C-terminal direction:
a segment (CDR-H1) having the amino acid sequence of SEQ ID NO: 6,
a segment (CDR-H2) having the amino acid sequence of SEQ ID NO: 7, and
a segment (CDR-H3) having the amino acid sequence of SEQ ID NO: 8 or having an amino acid sequence having 1 amino acid residue substitution compared to the amino acid sequence of SEQ ID NO: 8.

9) The protein according to any one of 4) to 8), wherein the protein is a single-chain antibody (scFv), an Fab fragment, an F(ab)₂ fragment, or an immunoglobulin (Ig); and/or
said protein is a fusion protein comprising a single-chain antibody (scFv), an Fab fragment, an F(ab)₂ fragment, or an immunoglobulin (Ig) as a first segment of the fusion protein and a second fusion protein segment.

10) The protein according to any one of 4) to 9), comprising an antibody light chain (subunit) and an antibody heavy chain (subunit),
said light chain comprising said light chain variable region and a light chain constant region, and
said heavy chain comprising said heavy chain variable region and at least one heavy chain constant region, preferably at least constant region C_{H}1.

11) The protein according to 10), wherein said heavy chain constant region comprises from 1, 2, or 3 Ig heavy chain constant domains, preferably three heavy chain constant domains.

12) The protein according to any one of 1) to 8), wherein said protein is an immunoglobulin selected from the group consisting of IgG, IgA, IgD, IgE, and IgM, or said protein is a fusion protein comprising said Ig and an additional fusion protein segment.

13) The protein according to 12), wherein the Ig is an IgG or is a fusion protein comprising an IgG and an additional fusion protein segment.

14) The protein according to any one of 1) to 13), comprising a light chain as follows:
(a) the amino acid sequence of said light chain is or comprises that of SEQ ID NO: 9, 10 or 11, or
(b) the amino acid sequence of said light chain is or comprises an amino acid sequence having 1 or 2 amino acid residue substitutions compared to the amino acid sequence of SEQ ID NO: 9, 10 or 11.

15) The protein according to any one of 4) to 14), wherein the light chain is a kappa light chain or a lambda light chain.

16) The protein according to any one of 4) to 15), comprising a heavy chain as follows:
(c) the amino acid sequence of said heavy chain is or comprises that of SEQ ID NO: 12, 13, 14, or 15, or
(d) the amino acid sequence of said heavy chain is or comprises an amino acid sequence having 1 or 2 amino acid residue substitutions compared to the amino acid sequence of SEQ ID NO: 12, 13, 14, or 15.

17) The protein according to any one of 4) to 16), wherein said protein is a fully human Ig.

18) The protein according to any one of 4) to 17), comprising an antibody heavy chain or Fc part incapable of binding to, or having reduced binding to, an Fc receptor or having a mutated constant region that reduces binding to an Fc receptor.

19) The protein according to 18), wherein the amino acid sequence of said heavy chain has at the position corresponding to position 334 of SEQ ID NO: 12 or 13 a P to G substitution for preventing or reducing binding of said protein to an Fc receptor.

20) The protein according to any one of 1) to 3) and 4) to 19), comprising a binding domain capable of binding to the human transferrin receptor 1 (hTfR1) for allowing crossing of said protein of the blood-brain-barrier, preferably said protein comprises a heavy chain having a modified C_{H}3 domain or comprises a C-terminal extension of the C_{H}3 domain that allows binding to the hTfR1.

21) The protein according to any one of 1) to 20), wherein said protein is capable of binding to human TREM2 via its variable region, preferably to the stalk region of hTREM2; generally, the protein or antibody is a hTREM2 agonist.

22) An antibody comprising an Ig light chain variable region as defined in 4) and an Ig heavy chain variable region as defined in 4).

23) The antibody according to 22), comprising:
a light chain wherein
(a) the amino acid sequence of said light chain is or comprises that of SEQ ID NO: 9, 10 or 11, or
(b) the amino acid sequence of said light chain is or comprises an amino acid sequence having 1 or 2 amino acid residue substitutions compared to the amino acid sequence of SEQ ID NO: 9, 10 or 11; and a heavy chain wherein:
(c) the amino acid sequence of said heavy chain is or comprises that of SEQ ID NO: 12, 13, 14, or 15, or
(d) the amino acid sequence of said heavy chain is or comprises an amino acid sequence having 1 or 2 amino acid residue substitutions compared to the amino acid sequence of SEQ ID NO: 12, 13, 14, or 15.

24) The antibody according to 22) or 23), comprising two light chains as follows:
(a) the amino acid sequence of said light chains is or comprises that of SEQ ID NO: 10 or 11, or
(b) the amino acid sequence of said light chains is or comprises an amino acid sequence having 1 or 2 amino acid residue substitutions compared to the amino acid sequence of SEQ ID NO: 10 or 11; and two heavy chains as follows:
(c) the amino acid sequence of said heavy chains is or comprises that of SEQ ID NO: 12, 13, 14, or 15, or
(d) the amino acid sequence of said heavy chains is or comprises an amino acid sequence having 1 or 2 amino acid residue substitutions compared to the amino acid sequence of SEQ ID NO: 12, 13, 14, or 15.

25) Pharmaceutical composition comprising the protein or antibody according to any one of 1) to 24) and a pharmaceutically acceptable carrier.

26) The protein or antibody according to any one of 1) to 24) or the pharmaceutical composition according to 25) for use in therapy or prevention.

27) The protein or antibody according to any one of 1) to 24) or the pharmaceutical composition according to 25) for use in a method of therapy or prevention of a neurodegenerative disease, such as Alzheimer's disease, preferably at an early stage patient or at an early stage of the disease of the patient.

28) The protein or antibody or pharmaceutical composition for the use according to 26) or 27), said use comprising parenteral administration of said protein or antibody to a mammal, preferably intravenous, subcutaneous or intraperitoneal administration.

29) Nucleic acid molecule encoding a protein, polypeptide, light chain and/or heavy chain as defined in any one of 1) to 24).

30) Nucleic acid molecule comprising a nucleotide sequence of SEQ ID NO: 25 or 26.

31) Eukaryotic cell comprising a protein according to any one of 1) to 24) or a nucleic acid molecule according to 29) or 30).

32) A method of treating or preventing of a neurodegenerative disease, such as Alzheimer's disease, comprising administering a protein or antibody as defined in any one of 1) to 24) or a pharmaceutical composition according to 25) to a mammal in need thereof.

The inventors have surprisingly identified antibodies that can strongly induce human TREM2/DAP-triggered SYK phosphorylation, which is the pivotal TREM2-dependent effector pathway in AD. SYK phosphorylation was up to 60-fold increased by antibody M07, whereas no or little activation was found with the other antibody clones, although M07 binds to the same epitope on the extracellular domain of TREM2 as H08 and M03.

Using phage display technology, the inventors have obtained fully human anti-TREM2 antibodies which were initially screened for antigen binding. The selected fully human IgG1-LALA modified antibodies were employed to determine the binding affinity to the extracellular domain of human TREM2, activation of human TREM2 signaling (human TREM2/DAP-dependent SYK phosphorylation), and most importantly, efficacy in a complex, relevant AD model using human brain cells which were differentiated from human induced pluripotent stem cells (hiPSC).

The LALA-modification strongly reduces effector function of Ig antibodies, notably of IgG and in particular IgG1 antibodies, which is important for studies with human immune and neuronal cells. The fully human backbones of the antibodies which we have generated are advantageous compared to existing humanized antibodies which are based on identification of clones in non-human animal immune systems (e.g., US2017240631A1 (Alector AL-002), and WO2020172450 A1 (Denali), because less immunological complications can be expected using fully human antibodies upon repeated preventive or therapeutic applications in vivo in humans.

Initially, the inventors identified a variety of structurally similar and related antibodies (heavy chain amino acid sequence homology 90% or more) which all bound to the extracellular (ec) domain of human TREM2 with high affinities (below 10-9 M). Much to the inventors' surprise, they then found that only one of these antibodies (antibody M07) could strongly induce human TREM2/DAP-triggered SYK phosphorylation, which is the pivotal TREM2-dependent effector pathway in AD. SYK phosphorylation was up to 60-fold increased by M07, whereas no or little activation was seen with the other antibody clones, although M07 binds to the same epitope on the extracellular domain of TREM2 as H08 and M03.

The increase in human TREM2/DAP-dependent pSYK level that was induced by M07 was much stronger than that observed for published as well as patented agonistic antibodies against human TREM2, and especially of any human anti-human TREM2 antibodies. The hT2AB antibody disclosed by AMGEN (WO2022120373 A1) was used in pSYK assays comparable to ours and caused a 12-fold increase over baseline (Ellwanger et al., 2021). Alector presented in its patent application (US2017240631 A1) numerous antibodies directed against human anti-TREM2. Phosphorylation of SYK was shown on protein level and a ~3-4 fold increase was reported for antibodies #22, #45 and #65 in human dendritic cells. In human macrophages, a 6-fold increase in SYK phosphorylation was observed. Denali presented several anti-hTREM2 antibodies in their patent application (WO2020172450 A1)-one of which is CL0020188. This antibody increased pSYK levels 4-fold compared to control antibody in TREM2-expressing HEK293 cells.

In addition, the inventors have used an innovative complex and relevant AD model using human brain cells which were differentiated from human induced pluripotent stem cells (hiPSC). None of the anti-TREM2 antibodies which had been known in the state of the art had been analyzed in a comparably sophisticated AD model using hiPSC-derived neurons and microglia. For instance, WO2020172450 A1 (Denali) discloses a phagocytosis assay using hiPSC-derived microglia and amyloid β. However, the analysis was not performed in co-culture with neurons. Therefore, the benefit of amyloid β phagocytosis on neurons cannot be determined. AD can also be studied in other disease models which all have inherent limitations. Most researchers still work in mouse models, which however often failed to predict clinical efficacy of anti-AD drug candidates.

The present invention solves the problem identified above and surprisingly provides fully human anti-human TREM2 antibodies which activate human TREM2-dependent human pSYK signaling so strongly that beneficial effects can be observed in a relevant AD model of human brain cells.

### BRIEF DESCRIPTION OF THE FIGURES

FIG. 1: Silver gel of human anti-TREM2 agonistic antibody M07. The first (left) lane shows non-reducing, non-boiling (NRNB) condition, whereas the second lane shows the sample after boiling in reducing buffer conditions. After reducing/boiling the antibody separates into light and heavy chains detected at 25 and 50 kDa.

FIG. 2: ELISA-based EC50 values of antibody binding to human ecTREM2: H01, H08, M03 and M07 bind to the human extracellular (ec) domain of TREM2 with similar EC50 values. M05 does not bind to ecTREM2. Mean values of N=2, technical replicates

FIG. 3: Cartoon of human TREM2 with highlighted epitope peptide within the stalk region. Modified from (Reifschneider et al., 2022). The amino acid sequence depicted is that of SEQ ID NO: 27.

FIG. 4: ELISA-based EC50 values of rat H01 and various human antibodies binding to an epitope peptide derived from the human TREM2 stalk domain: H01 and M05 do not bind to the peptide sequence present in the stalk region of human TREM2, whereas H08, M03 and M07 bind with a measured EC50 values between 329 and 832 μM. Mean values of N=2, technical replicates

FIG. 5: p-SYK signaling in HEK293-Flp-In hTREM2/hDAP12 upon antibody treatment (40 μg/ml): AlphaLISA assay for pSYK shows a significant activation of the signaling pathway upon addition of human anti-TREM2 antibodies H08, M03 and M07, and rat anti-TREM2 antibody H01. The activation is much stronger with antibody M07 (average of 36-fold increase over baseline) as compared to any other antibody tested. H05 and isotype controls for rat or human antibodies did not result in activation of the signaling pathway. Shown are means+/−SEM; One-way Anova with Brown-Forsythe post-hoc test; n=12 for H01 and H08, n=16 for M03, n=6 for M05, n=18 for MO7. **: p<0.01; ***: p<0.001; ****: p<0.0001.

FIG. 6: Titration of p-SYK signaling in HEK293-Flp-In hTREM2/hDAP12 upon antibody treatment: A. Titration curve of anti-TREM2 antibody H01 and M07 shows a much stronger activation of pSYK signaling with M07 at different antibody concentrations. B. Titration curve of anti-TREM2 antibody M07 shows a strong activation of pSYK signaling with M07 at different antibody concentrations. Shown are means+/−SEM; n=9; EC50=4.387 nM; KD=2.19 nM.

FIG. 7: iPSC-derived microglia neuron coculture model of Alzheimer's disease detects neurite degeneration and dead nuclei with amyloid β (AB). A. Timeline of the coculture of neurons and microglia. Addition of amyloid β to model Alzheimer's disease and parallel addition of TREM2 antibody to test for a neuroprotective effect. B. Representative images of the coculture containing microglia (Iba1) and neurons (β III Tubulin) in the control condition (medium) and 5 μM amyloid β condition. C. Detection of neurite degeneration and increasing numbers of dead nuclei with rising amyloid β doses. Shown are means+/−SD; n=6 technical replicates. D. Representative images from ICC staining for MAP2 and DAPI, and the Cellprofiler analysis of neurites and dead nuclei numbers.

FIG. 8: The anti-hTREM2 M07 antibody reduces amyloid β dependent neurite degeneration and cell death in a microglia neuron coculture system. A. Adding the anti-hTREM2 antibody M07 (0.6 M Aβ+M07 antibody) in addition to amyloid β significantly reduces neurite degeneration and dead nuclei numbers compared to only adding Aβ (0.6 μM Aβ) or Aβ together with an isotype control antibody (0.6 μM Aβ+Isotype antibody). B. Representative images of the ICC stainings for MAP2 and DAPI, and the Cellprofiler analysis of neurites and dead nuclei numbers. Shown are mean values+/−SD; n=6 technical replicates.

FIG. 9: Titration of p-SYK signaling in iPSC-derived microglia upon antibody treatment: titration curve of anti-TREM2 antibody M07 shows an activation of pSYK signaling with M07 at different antibody concentrations. n=1.

### DETAILED DESCRIPTION OF THE INVENTION

The protein and antibody of the invention can bind to the extracellular (ec) domain of human TREM2, notably to its stalk region. Further, the protein and antibody of the invention have excellent capability of activating human TREM2, notably activating p-SYK signaling. Accordingly, the protein and antibody is a TREM2 agonist, preferably an agonist of p-SYK signaling of hTREM2. Therefore, the protein and antibody of the invention are highly promising active agents for treating neurodegenerative disorders such as AD.

### Definitions

Herein, a protein is a monomeric protein, i.e. a protein comprising one subunit or polypeptide molecule, or a polymeric protein, i.e. a protein that comprises two or more subunits or polypeptide molecules. An example of a monomeric protein is a single-chain antibody (scFv) or a single-domain antibody. Examples of multimeric proteins are Fab fragments of an Ig, F(ab)₂ fragments of an Ig, or immunoglobulins that may be tetrameric. A protein may have modifications at side chains of amino acid residues, such as those described below for polypeptides.

A polypeptide is a polypeptide molecule, as opposed to a sequence stretch or moiety of a molecule. The amino acid residues of a polypeptide may have chemical modifications at the side chains of the residues, such as disulfide bonds between two cysteine residues of the same polypeptide or between two cysteine residues of different polypeptides. Other examples of chemical modifications of amino acid residue side chains is glycosylation, such as of asparagine residue of heavy chains, oxidized side chains, addition (linking) of markers, tags, labels, or other proteins or polypeptides. Modifications of amino acid residue side chains are not limited to moieties of small molecules, but may be other polypeptides or protein domains.

The term “region” of a protein or polypeptide refers to a domain of said protein or polypeptide, i.e. to an amino acid sequence stretch (or segment) of the polypeptide or protein. The polypeptide or protein comprises said region as an amino acid sequence stretch or segment of said polypeptide or protein. The protein or polypeptide comprises at least one amino acid residue more than the region, stretch or segment of it. The terms “region” and “domain” are used interchangeably herein.

The term “amino acid sequence” refer to the primary structure of a polypeptide, region, domain, segment or stretch. Amino acid sequences are frequently defined by referring to a reference sequence identified by a SEQ ID NO. Unless a subrange of a reference sequence is identified, a reference to a reference sequence is to the entire amino acid sequence of the reference sequence.

A (polypeptide) stretch or segment refers to a plurality of (contiguous) amino acid residues within a polypeptide molecule, the polypeptide molecule comprising more amino acid residues than the stretch or segment.

The term “antibody” refers to a protein with an immunoglobulin fold that specifically binds to an antigen via its variable region(s), here to the extracellular (ec) domain of human TREM2, notably to the epitope of SEQ ID NO: 16. The term encompasses polyclonal antibodies and monoclonal antibodies, single-domain antibodies, heavy chain antibodies, single-chain antibodies. The term “antibody” as used herein, also includes Ig fragments that retain binding specificity via its variable regions, including but not limited to Fab, F(ab′)₂, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively. Herein, preferred antibodies are immunoglobulins (igs). The antibody as well as the Ig for use in the invention is preferable monoclonal and, further, fully human.

An immunoglobulin (Ig) is a protein of the globulin-type (naturally found in serum or other body fluids) that possesses antibody activity, i.e. specifically binds to an antigen via its variable region(s). An Ig molecule comprises two light (L) and two heavy (H) chains (or polypeptide chains or subunits) linked together by disulfide bonds. An Ig may form an oligomeric structure, such as IgM which is pentameric Ig. Igs are divided into the five classes IgG, IgM, IgA, IgD and IgE based on antigenic and structural differences in the H chains. An Ig region is a domain of an Ig. The domains of an Ig are the variable domains and the constant domains. An Ig heavy chain has one variable domain (or region) and three different constant domains referred as C_{H}1, C_{H}2, and C_{H}3. An Ig light chain has two domains, the variable domain and a constant domain.

The term “light chain” or “antibody light chain” means a polypeptide comprising an immunoglobulin (Ig) light chain (LC) variable region (or domain) and an Ig light chain constant region (or domain).

The term “heavy chain” or “antibody heavy chain” means a polypeptide comprising an Ig heavy chain (HC) variable region and at least one Ig constant region, generally at least a C_{H}1 region (or domain). Preferably, a heavy chain or antibody heavy chain means a (full) Ig heavy chain comprising an Ig heavy chain variable region (or domain) and three Ig constant regions (or domains) C_{H}1, C_{H}2, and C_{H}3.

### Protein and Antibody of the Invention

The protein of the invention comprises or consists of an (Ig) heavy chain variable region, wherein the amino acid sequence of the heavy chain variable region is that of SEQ ID NO: 2 or is an amino acid sequence having 1 or 2 amino acid residue substitutions compared to SEQ ID NO: 2. Alternatively, the protein of the invention comprises or consists of an (Ig) light chain variable region, wherein the amino acid sequence of the light chain variable region is that of SEQ ID NO: 1 or is an amino acid sequence having 1 or 2 amino acid residue substitutions compared to SEQ ID NO: 1. An example of such protein is a single-domain antibody or nanobody. A single-domain antibody consists of a variable domain of either a heavy or a light chain, preferably a heavy chain.

In another embodiment, the protein of the invention may be a heavy chain antibody. In such embodiment, the protein generally comprises or consists of a (one or preferably two) heavy chain(s), (each) comprising the heavy chain variable region, the amino acid sequence of the heavy chain variable region being that of SEQ ID NO: 2 or being an amino acid sequence having 1 or 2 amino acid residue substitutions compared to SEQ ID NO: 2. The heavy chain preferably further comprises, apart from the variable region, at least one constant region (domain), such as two constant regions or five constant regions. The protein may comprise or consist of two (preferably identical) subunits or polypeptides, each polypeptide comprising said variable region and at least one, preferably at least two, heavy chain constant regions. An example of such heavy chain antibody is a V_{H}H antibody (camelid-type). Alternatively, the protein may comprise or consist of two (preferably identical) subunits or polypeptides, each polypeptide comprising said variable region and five heavy chain constant regions. An example is a V_{NAR} antibody (cartilaginous fish-type). In all these embodiments, the CDRs of the heavy chain variable domains are as described below.

In preferred embodiments described in the following, the protein of the invention comprises a light chain variable region and a heavy chain variable region.

Accordingly, the protein of the invention may comprise a light chain variable region and a heavy chain variable region, wherein the amino acid sequence of the light chain variable region is that of SEQ ID NO: 1 or is an amino acid sequence having 1 or 2 amino acid residue substitutions compared to SEQ ID NO: 1, and
the amino acid sequence of the heavy chain variable region is that of SEQ ID NO: 2 or is an amino acid sequence having 1 or 2 amino acid residue substitutions compared to SEQ ID NO: 2.

Preferably, the protein comprises an antibody light chain and an antibody heavy chain. The antibody light and heavy chains are polypeptides that comprise the light chain variable region as defined above and the heavy chain variable region as defined above, respectively. The variable region (both of the light and heavy chain) comprises three hypervariable regions, generally referred to as complementarity determining regions (CDRs) and numbered CDR1 to CDR3 in N-terminal to C-terminal direction. The CDRs are preceded and separated by regions of little variability, generally referred to as framework regions (FRs) and numbered FR1 to FR4 in N-terminal to C-terminal direction of the chains.

The light chain (LC) variable region may comprise, preferably in CDR-L3, a segment of the amino acid sequence of SEQ ID NO: 5 or of an amino acid sequence having 1 amino acid residue substitution compared to the amino acid sequence of SEQ ID NO: 5; and the heavy chain (HC) variable region may comprise, preferably in CDR-H3, a segment of the amino acid sequence of SEQ ID NO: 8 or of an amino acid sequence having 1 amino acid residue substitution compared to the amino acid sequence of SEQ ID NO: 8. Absence of such amino acid substitutions is preferred for both these CDRs and chains in this and the following embodiments.

Preferably, the light chain variable region comprises, preferably in CDRs L1 and L3, the following amino acid segments in N-terminal to C-terminal direction:
a segment (CDR-L1) of the amino acid sequence of SEQ ID NO: 3, and
a segment (CDR-L3) of the amino acid sequence of SEQ ID NO: 5 or an amino acid sequence having 1 amino acid residue substitution compared to the amino acid sequence of SEQ ID NO: 5; and
the heavy chain (HC) variable region comprises, preferably in CDRs H2 and H3, the following amino acid segments in N-terminal to C-terminal direction:
a segment (CDR-H2) of the amino acid sequence of SEQ ID NO: 7, and
a segment (CDR-H3) of the amino acid sequence of SEQ ID NO: 8 or of an amino acid sequence having 1 amino acid residue substitution compared to the amino acid sequence of SEQ ID NO: 8.

More preferably, the light chain (LC) variable region comprises, preferably in CDRs L1 to L3, the following amino acid segments in N-terminal to C-terminal direction:
a segment (CDR-L1) of the amino acid sequence of SEQ ID NO: 3,
a segment (CDR-L2) of the amino acid sequence of SEQ ID NO: 4, and
a segment (CDR-L3) of the amino acid sequence of SEQ ID NO: 5 or of an amino acid sequence having 1 amino acid residue substitution compared to the amino acid sequence of SEQ ID NO: 5; and
the heavy chain (HC) variable region comprises, preferably in CDRs H1 to H3, the following amino acid sequence segments in N-terminal to C-terminal direction:
a segment (CDR-H1) of the amino acid sequence of SEQ ID NO: 6,
a segment (CDR-H2) of the amino acid sequence of SEQ ID NO: 7, and
a segment (CDR-H3) of the amino acid sequence of SEQ ID NO: 8 or of an amino acid sequence having 1 amino acid residue substitution compared to the amino acid sequence of SEQ ID NO: 8.

Preferred are CDRs not having an amino acid residue substitution compared to the reference sequences given. As explained above, these segments (CDRs) are generally not present in said chains or regions contiguously, but separated by framework regions, as can be seen from the SEQ ID NO: 1 and 2.

The protein of the invention may be a single-chain antibody (scFv). In this case, the protein is a polypeptide comprising or consisting of a light chain variable region and a heavy chain variable region in this or the opposite order in N-terminal to C-terminal direction. The variable regions are as defined above and the CDRs are preferable also as described above. An scFv normally does not contain constant regions.

However, apart from the variable regions, the light and heavy chains of the protein of the invention generally further comprise one or more Ig constant domains or all domains of the respective constant regions of (full) Ig light and heavy chains. Thus, the protein may comprise an antibody light chain and an antibody heavy chain, wherein:
the light chain comprises a light chain variable region, the amino acid sequence of said light chain variable region being that of SEQ ID NO: 1 or being an amino acid sequence having 1 or 2 amino acid residue substitutions compared to SEQ ID NO: 1, and
the heavy chain comprises a heavy chain variable region, the amino acid sequence of the heavy chain variable region being that of SEQ ID NO: 2 or being an amino acid sequence having 1 or 2 amino acid residue substitutions compared to SEQ ID NO: 2. Embodiments without one or none (i.e. with no) amino acid residue substitution are preferred. The CDRs are preferable as described above.

As the light chain of natural immunoglobulins contains one Ig constant domain or region, the light chain of the protein of the invention preferably contains a (notably one) constant region. The light chain may be a kappa light chain or a lambda light chain, the former being preferred. As the heavy chain of natural immunoglobulins contains three constant domains (generally referred to as C_{H}1, C_{H}2 and C_{H}3), the heavy chain of the protein of the invention thus generally contains at least one constant domain, preferably a C_{H}1 domain. In one embodiment, the heavy chain contains two (Ig) constant domains, preferably a C_{H}1 and a C_{H}2 domains. Even more preferably, the heavy chain of the protein of the invention comprises three (Ig) constant domains, such as C_{H}1, C_{H}2 and C_{H}3 domains (in N-terminal to C-terminal direction).

The protein of the invention may thus be an Fab fragment of an Ig, i.e. it may comprise a light chain and a heavy chain; the former comprising or consisting of a polypeptide comprising a light chain (kappa or lambda), the latter comprising or consisting of a heavy chain variable region and a, or one, (Ig) heavy chain constant region, generally C_{H}1. The light and heavy chain are generally covalently linked by a disulfide bridge.

The protein of the invention may, alternatively, be an F(ab)₂ fragment of an Ig, comprising two Fab fragments linked by one or more disulfide bridges.

The protein of the invention may comprise an Ig light chain and a (i.e. full or complete) Ig heavy chain. Full heavy chain means that the heavy chain comprises, apart from the variable domain, three Ig constant domains. Accordingly, the heavy chain preferably comprises, apart from the variable domain, three constant domains C_{H}1, C_{H}2 and C_{H}3. In a more preferred embodiment, the protein of the invention comprises two light chains and two (full) heavy chains. Accordingly, the protein is preferably an immunoglobulin of any isotype, such as IgG, IgM, IgA, IgD and IgE. Preferably, it is an IgG. The Ig comprises two (generally identical) Ig light chains and two Ig heavy chains that are generally (but not necessarily) identical. Among IgGs, the protein may be, depending on the heavy chains, an IgG1, IgG2, IgG3 or IgG4. Preferably, it is an IgG1, IgG2 or IgG3, and more preferably an IgG1 as, for example, the clone M07 described and use in the Examples.

A preferred protein or antibody of the invention comprises a light chain, wherein
(a) the amino acid sequence of said light chain is or comprises that of SEQ ID NO: 9, 10 or 11, preferably SEQ ID NO: 10, or 11; or
(b) the amino acid sequence of said light chain is or comprises an amino acid sequence based on a kappa-1 light chain backbone having 1 or 2 amino acid residue substitutions compared to the amino acid sequence of SEQ ID NO: 9 or 10, preferably SEQ ID NO: 10; or
(c) the amino acid sequence of said light chain may also be or may comprise an amino acid sequence based on a lambda light chain backbone having 1 or 2 amino acid residue substitutions compared to the amino acid sequence of SEQ ID NO: 11.

In the above embodiments, notably those of (a), (b) and (c), the protein or antibody preferably further comprises a heavy chain, wherein:
(d) the amino acid sequence of said heavy chain is or comprises that of SEQ ID NO: 12, 13, 14, or 15, preferably of SEQ ID NO: 13 or 15, or
(e) the amino acid sequence of said heavy chain is or comprises an amino acid sequence having 1 or 2 amino acid residue substitutions compared to the amino acid sequence of SEQ ID NO: 12, 13, 14, or 15, preferably of SEQ ID NO: 13 or 15.

It is further preferred in the above embodiments that the protein or antibody is an immunoglobulin comprising two identical (Ig) light chains and two (Ig) heavy chains (that may also be identical), and more preferably is an IgG1 antibody. The protein or antibody may thus comprise or consist of two light chains, wherein
(a) the amino acid sequence of said light chains is or comprises that of SEQ ID NO: 9, 10 or 11, preferably SEQ ID NO: 10, or
(b) the amino acid sequence of said light chains is or comprises an amino acid sequence having 1 or 2 amino acid residue substitutions compared to the amino acid sequence of SEQ ID NO: 9, 10 or 11, preferably SEQ ID NO: 10, and two heavy chains, wherein:
(c) the amino acid sequence of said heavy chains is or comprises that of SEQ ID NO: 12, 13, 14, or 15, preferably of SEQ ID NO: 13 or 15, or
(d) the amino acid sequence of said heavy chains is or comprises an amino acid sequence having 1 or 2 amino acid residue substitutions compared to the amino acid sequence of SEQ ID NO: 12, 13, 14, or 15, preferably of SEQ ID NO: 13 or 15. The two light chains and the two heavy chains are preferably identical in amino acid sequence. The embodiments with one or none amino acid residue substitution are preferred. The CDRs are as defined above.

Further embodiments of the protein or antibody of the invention are as follows: the protein or antibody comprises two subunits (chains) of the kappa-1 light chains of SEQ ID NO: 9 or of SEQ ID NO: 10, the latter being preferred, or lambda light chains of SEQ ID NO: 11, and two subunits of the heavy chains of SEQ ID NO: 12, SEQ ID NO: 13, SEQ ID NO: 14 or SEQ ID NO: 15, preferably of SEQ ID NO: 13 or 15.

In another embodiment, the protein comprises two light chains of SEQ ID NO: 10, one heavy chain of SEQ ID NO: 13 and one heavy chain of SEQ ID NO: 15. In a further embodiment, the protein comprises two light chains of SEQ ID NO: 10 and two heavy chains of SEQ ID NO: 13. In a further embodiment, the protein comprises two light chains of SEQ ID NO: 10 and two heavy chains of SEQ ID NO: 15. In these embodiments, the heavy chains may have 1 or 2 amino acid residue substitutions in sequence portions outside the heavy chain CDRs defined above.

In still another embodiment, the protein comprises two light chains of SEQ ID NO: 11, one heavy chain of SEQ ID NO: 13 and one heavy chain of SEQ ID NO: 15. In a further embodiment, the protein comprises two light chains of SEQ ID NO: 11 and two heavy chains of SEQ ID NO: 13. In a further embodiment, the protein comprises two light chains of SEQ ID NO: 11 and two heavy chains of SEQ ID NO: 15. In these embodiments, the heavy chains may have 1 or 2 amino acid residue substitutions in sequence portions outside the heavy chain CDRs defined above.

As indicated above, the protein of the invention is preferably an antibody, more preferably and Ig, comprising a (Ig) light chain variable region as defined above and a (Ig) heavy chain variable region as defined above. The antibody comprises preferably a light chain wherein:
(a) the amino acid sequence of said light chain is or comprises that of SEQ ID NO: 9 or 10, or
(b) the amino acid sequence of said light chain is or comprises an amino acid sequence having 1 or 2 amino acid residue substitutions compared to the amino acid sequence of SEQ ID NO: 9 or 10; preferably 10, and
   a heavy chain wherein:
(c) the amino acid sequence of said heavy chain is or comprises that of SEQ ID NO: 12, 13, 14, or 15, preferably SEQ ID NO: 13 or 15, or
(d) the amino acid sequence of said heavy chain is or comprises an amino acid sequence having 1 or 2 amino acid residue substitutions compared to the amino acid sequence of SEQ ID NO: 12, 13, 14, or 15, preferably SEQ ID NO: 13 or 15.

Preferred embodiments are analogous to those given above for the protein of the invention.

Alternatively, the protein of the invention may be an antibody, preferably and Ig, comprising a (Ig) light chain variable region as defined above and a (Ig) heavy chain variable region as defined above. The antibody comprises preferably a light chain wherein:
(a) the amino acid sequence of said light chain is or comprises that of SEQ ID NO: 11, or
(b) the amino acid sequence of said light chain is or comprises an amino acid sequence having 1 or 2 amino acid residue substitutions compared to the amino acid sequence of SEQ ID NO: 11 and a heavy chain wherein:
(c) the amino acid sequence of said heavy chain is or comprises that of SEQ ID NO: 12, 13, 14, or 15, preferably of SEQ ID NO: 13 or 15, or
(d) the amino acid sequence of said heavy chain is or comprises an amino acid sequence having 1 or 2 amino acid residue substitutions compared to the amino acid sequence of SEQ ID NO: 12, 13, 14, or 15, preferably SEQ ID NO: 13 or 15.

Preferred embodiments are analogous to those given above for the protein of the invention.

The protein of the invention or a polypeptide of said protein may be a fusion protein. The fusion protein comprises a polypeptide comprising, as a first segment, any of the chains, regions or domains described above and a second segment (preferably C-terminal to the first segment). The second fusion protein segment may provide the protein or antibody with an additional functionality, such as binding to a receptor (other than hTREM2). The fusion protein may be a fusion protein comprising an Ig selected from IgG, IgA, IgD, IgE, and IgM, and a second fusion protein segment, whereby fusion proteins comprising IgGs are preferred. Preferably, the fusion protein is a fusion protein of any such Ig, wherein the fusion protein comprises two Ig light chains and two heavy chains, wherein the polypeptide of at least one, preferably both, heavy chain(s) is a fusion protein comprising the second fusion protein segment.

The protein or antibody of the invention may have further modifications to facilitate transport of the protein or antibody into the brain of a subject. For the case of parenteral, such as intravenous administration, the protein or antibody may have a modification that facilitates crossing of the blood brain barrier (BBB). Crossing of the BBB and brain delivery of the protein or antibody is known in the art, for a review see Pardridge (2015), Expert Opinion on Drug Delivery, 12:2, 207-222 (DOI: 10.1517/17425247.2014.952627). Established solutions exploit binding of the protein to be delivered to the human transferrin receptor (hTfR). WO2018152285 and WO2018152326 of Denali Therapeutics and Kariolis et al. (Science Translational Medicine, Vol. 12, No. 545; DOI: 10.1126/scitranslmed.aayl359) describe amino acid residue substitutions in the CH3 region of an antibody heavy chain to achieve specific binding to the TfR. WO2014033074 and WO2015101588 (Roche) describe BBB shuttle modules comprising a brain effector entity, a linker and a monovalent binding entity that binds to the BBB receptor such as TfR.

Accordingly, the protein or antibody of the invention may comprise a binding domain capable of binding to the hTfR1 for allowing crossing of said protein of the BBB, preferably said protein or antibody comprises a heavy chain having a modified CH3 domain that binds to the hTfR1. The heavy chain CH3 domain may be modified as described in WO2018152285, WO2018152326 or Kariolis et al. Alternatively, for analogous purposes, the protein of the invention may comprise a fusion protein as described above.

For treating or preventing neurological disorders or diseases, the protein or antibody of the invention may have reduced Fc effector functions for preventing undesired or unnecessary effects on the immune system. For this purpose, the protein of the invention may lack the CH2 and CH3 domain of antibody heavy chains. Preferably, however, the protein comprises heavy chains including CH2 and CH3 domains, but the CH2 domain has one or more amino acid residue substitutions that reduce Fc effector functions. An example of such mutation is the well-known L234A and L235A double mutation described inter alia in the review of Wang et al. (Protein Cell 2018, 9 (1), 63-73; doi.org/10.1007/s13238-017-0473-8) for reducing binding of said protein to an Fc receptor and thus reducing effector function. Corresponding mutations are generally preferred for all embodiments of the present invention wherein the protein contains a heavy chain with C_{H}2 domain. Each of SEQ ID NOs: 12 to 15 contains this double mutation at its corresponding position, at the position of 239 and 240 of SEQ ID NO: 13. Additionally, for further reducing effector function of the protein or antibody, the heavy chain may further have at the position corresponding to position 334 of SEQ ID NO: 13 or 15 a P to G substitution (leading to the LALA-PG triple mutation) for preventing or reducing binding of said protein to an Fc receptor. This mutation is described in detail in WO2012130831 A1.

### Nucleic Acid Molecules

The invention provides a nucleic acid molecule encoding a protein, polypeptide, light chain and/or heavy chain as defined above. The nucleic acid molecule may be a plasmid or vector comprising one or more constructs or cistrons encoding the protein, polypeptide, light chain and/or heavy chain and regulatory genetic elements for expressing them in suitable cells. In embodiments wherein the protein comprises two or more different polypeptide molecules, the plasmid or vector may comprise two or more constructs or cistrons, one for each polypeptide to be expressed. Alternatively, the invention provides a kit to two nucleic acid molecule, one encoding a first polypeptide and one encoding a second polypeptide of the protein of the invention. The nucleic acid molecule may comprise a nucleotide sequence of SEQ ID NO: 25 or 26.

### Cells

The invention provides a cell, preferably a eukaryotic cell, comprising a protein according to the invention or a nucleic acid molecule according to the invention such as those described above as described above. The cell is preferably used for producing and expressing the protein of the invention. For use of the protein in humans, the cell is preferably a human cell in order to endow the protein or antibody with human-like glycosylation. However, the cell may have a genetically engineered glycosylation machinery to endow the protein with the desired glycosylation.

### Production of the Protein or Antibody

The protein or antibody of the invention may be expressed in a suitable expression system from a nucleic acid molecule encoding it, as generally known in the art. In the case of hetero-oligomeric proteins such as immunoglobulins, the light and heavy chains may be expressed in the same cell, preferably eukaryotic cell, from a bi-cistronic plasmid as described in the examples. The light and heavy chains may be expressed in the form containing N-terminal leader sequences directing secretion of the leaders. The leaders should be such that they are cleaved off after secretion or in the secretory pathway of the cell, whereupon the light and heavy chains can assemble to form the oligomeric protein, preferably without the leader sequences. For use in humans, the cell system used for expression is preferably human in order to endow the protein or antibody with human-like glycosylation if desired.

### Pharmaceutical Compositions and Formulations

The invention also provides a pharmaceutical composition comprising the protein or antibody of the invention. The composition generally further comprises one or more pharmaceutically acceptable carriers and/or excipients. A pharmaceutically acceptable carrier includes any solvents or dispersion media that are physiologically compatible and that does not interfere with or otherwise inhibit the activity of the active agent. The preferred solvent is water that may additionally contain excipients.

Examples of excipients are carbohydrates, such as glucose, sucrose, or dextran, antioxidants, such as ascorbic acid or glutathione, chelating agents, stabilizers, and/or buffers. The pharmaceutical composition can be manufactured by mixing the protein or antibody in a manner that is known to those of skill in the art, e.g., by means of conventional mixing, dissolving, or lyophilizing processes.

For the desired parenteral administration, the pharmaceutical composition may be administered as a solution, generally by injection or infusion. For injection, the protein or antibody can be formulated into preparations by dissolving, suspending or emulsifying them in an aqueous solvent that may contain conventional additives such as solubilizers, isotonic agents, suspending agents, emulsifying agents, stabilizers and preservatives. In some embodiments, compounds can be formulated in aqueous solutions, e.g, in physiologically compatible buffers such as physiological saline buffer. Formulations for injection can be presented in unit dosage form, e.g, in ampules or in multi-dose containers, with or without an added preservative. The compositions can take such forms as suspensions, solutions or emulsions in oily or aqueous vehicles.

However, the pharmaceutical composition may, alternatively, be solid composition, e.g. in lyophilized form. The solid form may be reconstituted before use with a suitable solution or medium, as described above.

Typically, the pharmaceutical composition for use in in vivo administration is sterile. Sterilization can be accomplished according to methods known in the art, e.g., sterile filtration of solution or by irradiation.

### Therapy or Prevention

The protein or antibody of the invention are used in therapy or prevention. The disorder of disease to be prevented or treated is a neurodegenerative disease, such as Alzheimer's disease. For treating or preventing the disorder or disease in a patient, the protein or antibody is administered to a subject in need of the treatment/prevention. The subject or patient is a mammal, preferably a human. The invention also provides a method of treating or preventing of a neurodegenerative disease such as Alzheimer's disease in a mammal, preferably a human, comprising administering a protein or antibody of the invention or the pharmaceutical composition to the mammal or human in need thereof.

In the therapy or prevention of a neurodegenerative disease, the patient to which the protein or antibody is administered, is preferably a patient at an early stage of the neurodegenerative disease, such as Alzheimer's disease, since efficacy at an early stage is expected to be higher than in an advanced stage of the disease. Treating at an early stage thus prevents or inhibits progression of the disease to a more advanced or severe stage.

The stage of a neurodegenerative disease, such as Alzheimer's disease, may be determined by established methods. One such method is the mini-mental state examination (MMSE) or Folstein test that is based on a 30-point questionnaire that is used extensively in clinical and research settings to measure cognitive impairment. The MMSE may be used in the version described by Tombaugh, Tom N.; Mcintyre, Nancy J. (1992). “The Mini Mental Status Examination: A comprehensive review”. Journal of the American Geriatrics Society. 40 (9): 922-935. doi: 10.1111/j.1532-5415.1992.tb01992. The MMSE is commonly used in medicine to screen for dementia. It is also used to estimate the severity and progression of cognitive impairment and to follow the course of cognitive changes in an individual over time. Thus, the MMSE is an effective way to document an individual's response to treatment. Any score of 24 or more (out of 30) indicates a normal cognition. Below this, scores can indicate severe (≤9 points), moderate (10-18 points) or mild (19-23 points) cognitive impairment. The raw score may also need to be corrected for educational attainment and age. Low to very low scores correlate closely with the presence of dementia, although other mental disorders can also lead to abnormal findings on MMSE testing. The presence of purely physical problems can interfere with interpretation if not properly noted; for example, a patient may be physically unable to hear or read instructions properly or may have a motor deficit that affects writing and drawing skills.

Another method for determining the stage of a neurodegenerative disease, such as Alzheimer's disease, is the CDR Global Score (Clinical Dementia Rating Scale). The CDR is a global rating scale for staging patients diagnosed with dementia. The CDR evaluates cognitive, behavioral, and functional aspects of Alzheimer disease and other dementias. Rather than a mental status examination or inventory, the rater makes a judgment on six categories based on all the information available. The scoring system for the CDR is heavily dependent on the memory scores, but the CDR has good inter-rater reliability in staging dementia. This CDR is a widely used scale in both Alzheimer disease centers and dementia research. CDR is estimated on the basis of a semistructured interview of a subject and a caregiver (informant) and on the clinical judgment of the clinician. CDR is calculated on the basis of testing six different cognitive and behavioral domains such as memory, orientation, judgment and problem solving, community affairs, home and hobbies performance, and personal care. The CDR is based on a scale of 0-3: no dementia (CDR=0), questionable dementia (CDR=0.5), MCI (CDR=1), moderate cognitive impairment (CDR=2), and severe cognitive impairment (CDR=3). Two sets of questions are asked, one for the informant and another for the subject. The set for the informant includes questions about the subject's memory problem, judgment and problem solving ability of the subject, community affairs of the subject, home life and hobbies of the subject, and personal questions related to the subject. The set for subject includes memory-related questions, orientation-related questions, and questions about judgment and problem-solving ability. The method is described in Handbook of Clinical Neurology, Volume 167, 2019, Pages 89-104, Chapter 6-Cognitive and neuropsychological examination of the elderly.

Accordingly, the invention provides a protein or antibody for the use in the treatment or prevention of a neurodegenerative disease, such as Alzheimer's disease, of a patient having a early stage of the disease, preferably as follows:
at a score of 23 or lower, preferably at a score of from 10 to 23, more preferably at a score of from 19 to 23, cognitive impairment of the patient in the mini-mental state examination (MMSE) test, or
at a score of 0.5 or higher and 2 or lower cognitive impairment of the patient on the Clinical Dementia Rating Scale (CDR Global Score).

The protein or antibody is preferably administered parenterally. Examples of preferred administration routes are intravenous, subcutaneous and intraperitoneal administrations.

The protein or antibody may be administered to a subject at a therapeutically effective amount or dose. A dose range per administration is of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the frequency of administration, chosen route of administration, the formulation of the composition, patient response, the severity of the condition, and the judgment of the prescribing physician. The dosage can be increased or decreased over time, as required by an individual patient. A patient initially may be given a low dose, which is then increased to an efficacious dosage tolerable to the patient. Determination of an effective amount is well within the capability of those skilled in the art.

The protein or antibody may be administered at a frequency of once every one to 6 weeks, preferably once every 2 to 4 weeks.

### Examples

The present invention is not limited to the examples described in the following.

### I. Materials and Methods

### Identification of Antibody Clones

A part of the human TREM-2 extracellular domain was used as coated antigen in phage display screens of phage libraries which contain a full repertoire of human antibody sequences (diversity of at least 5×10¹⁰ clones), and were carried out at Proteogenix, Strasbourg, France. Several phage binders were identified which bound to the antigen with high affinity. Using standard techniques, human DNA and protein sequences of the respective variable regions of light and heavy chains were identified from monoclonal phage preparations. Clones were specifically modified for optimized codon using input from other partners. Each of these heavy chain variable domain sequences were used to synthesize full human IgG1 heavy chains by combining with constant domain sequences (gene accession no. UniProtKB-PODOX5), which included mutations of L to A at the appropriate positions 239/240 (corresponding to the consensus sequence positions 234 and 235) by synthetic gene assembly at GeneArt, Regensburg, using further codon optimization for Cricetus griseus. In addition, the respective light chain variable domains were combined with the constant region sequences of kappa-1 light chains (accession no. UniProtKB—P0DOX7) were assembled similarly. Restriction sites for AvrII were added at the beginning of the heavy chain genes, and for BstZ171 at the end. Similarly, restriction sites for Eco RV were added at the beginning of the light chain genes, and for PacI at the end. These DNA fragments were then used to insert heavy and light chain genes into the respective multiple cloning sites (AvrII-BstZ171 and Eco RV-PacI) of the bi-cistronic vector pCHOv1 which is part of the FreedomTS CHO-S kit (Thermo Fisher cat #A13696-01). Resulting vectors were generated at GeneArt, Regensburg, and subjected to full quality control, and purified using columns. This plasmid allows for expression of two different proteins in one cell under two hybrid modifications of cytomegalovirus (CMV) promotors, especially for the expression of antibody heavy and light chains in one cell such as chinese hamster ovary cells (CHO).

An IgG1-LALA isotype control was created using the heavy and light chain sequences of an anti-green fluorescent protein (GFP) antibody.

E. coli DH5α were transformed with 1 ng of plasmid DNA. Transformed bacteria were strike out on agar plates with 50 μg/mL kanamycin and incubated at 37° C. over night. The next day, a colony was picked and grown in LB medium containing 50 μg/mL kanamycin and incubated at 37° C. overnight. Maxi Prep was performed with NucleoBond Xtra Maxi EF, Maxi kit for endotoxin-free plasmid DNA (Machery Nagel cat #740424.50).

The coding sequences of the light and heavy chains of clone M07 are given in SEQ ID NO: 25 and 26, respectively.

The term “clone M07” refers to an IgG1 antibody of the invention expressed from coding sequences encoding the kappa-1 light chain of SEQ ID NO: 9 and encoding the heavy chain of SEQ ID NO: 12. Upon secretion, the N-terminal leaders are cleaved off. Thus, the mature light chain has the amino acid sequence of SEQ ID NO: 10 and the mature heavy chain has the amino acid sequence of SEQ ID NO: 13. The experiments described in the following are performed with antibody M07 comprising two of said mature light chains and two of said heavy chains. The heavy chain polypeptides of SEQ ID NO: 12 and 13 contain in the C_{H}3 domains the binding segment (TFN) for binding to the human transferrin receptor (hTfR). SEQ ID NO: 14 and 15 are heavy chains corresponding to SEQ ID NO: 12 and 13, but lack the binding segment (TFN) in the C_{H}3 domain and have the corresponding wild-type sequences instead of the TFN. All heavy chains contain the LALA double mutation.

### HEK293-Flp-In Cell Culture

HEK293-Flp-In cells were cultured in Dulbecco's modified Eagle's medium (DMEM) with GlutaMAX I, supplemented with 10% (v/v) fetal calf serum (FCS), 1% (v/v) penicillin/streptomycin and 0.4% (v/v) Hygromycin B.

### CHO-S Kit and Gene Transfer, Selection and Purification

CHO-S cells, which are commercially available as part of the FreedomTS CHO-S kit (Thermo Fisher cat #A13696-01), were thawed and grown in cell culture as recommended in the manufacturer's instructions using recommended media. Gene transfer of pCHOv1 plasmids containing the respective heavy and light chain sequences under the control of two different hybrid CMV promotors was carried out according to manufacturer's instructions. After positive selection of successfully transfected CHO-S cells, cells were kept in culture under selection pressure as recommended in the manufacturer's instructions. From time to time, supernatant was harvested and prepared for antibody purification. In brief, supernatant (~250 ml) was diluted 1:4 with binding buffer (20 mM sodium phosphate; pH=7.0) and loaded onto a 1 ml Protein A column (Cytiva cat #17040201) with the help of a peristaltic pump. Next, the column was washed with 10 column volumes of binding buffer and the antibody was eluted with elution buffer (0.1 M glycine-HCl; pH=2.7) into neutralization buffer (1 M TRIS-HCl, pH=9). Fractions containing protein (checked via Nano-Drop) were pooled and dialysed against 1×PBS overnight. Dialysed sample was concentrated and the concentration was determined via Nano-Drop.

Differentiation of hiPSC-Derived Microglia (hiMGL)

We differentiated hiMGL from iPSCs as described (Abud et al., 2017) with modifications to improve efficiency and yield: When iPSCs were 70-90% confluent, they were split 1:100-200 onto GelTrex-coated 6-well plates for the HPC differentiation using EDTA to get around ~30 small colonies per well. Cells were fed with 2 ml of HemA medium (HPC differentiation kit, StemCell Technologies) on day 0 and half-fed with 1 ml on day 2. Media was switched to 2 ml of HemB on day 3 with half-feeds on days 5 and 7 and 1 ml added on top on day 10. On day 12 HPCs were collected as non-adherent cells to either freeze or continue with the microglia differentiation. HPCs were frozen at 1 million cells per mL in BamBanker (Wako). They were then thawed directly onto GelTrex-coated 6-well plates with 1 million cells evenly distributed among 6 wells in 2 ml iMGL media with 25 ng/ml M-CSF, 100 ng/ml IL-34, and 50 ng/ml TGF-β added fresh. 1 ml of media was added on top every other day. During the microglia differentiation, the cells were split 1:2 every 6-8 days depending on confluency. A very similar differentiation protocol was published recently (McQuade et al., 2018). We did not use CD200 and CX3CL1 as this did not seem to have an effect on hiMGL gene expression, as determined by Nanostring analysis (data not shown). hiMGL were used for experiments on day 16 of the differentiation.

Differentiation of hiPSC-Derived Cortical Neurons

For differentiation of cortical neurons, first neural precursor cells (NPCs) were differentiated from hiPSC and NPCs and were further differentiated towards cortical neurons as described (Gregg et al., 2016) with modifications. Specifically, hiPSC were grown until 100% confluency on GelTrex coated plates. Neural induction (NI) medium, containing 10 UM SB431542 and 250 nM LDN193189, was added to the cells (day in vitro 0 (DIVO)) and maintained for 12 days. Cells were split as single cells using Accutase on ~DIV2 and ~DIV8 using ROCK inhibitor. On DIV2 0.26 mio cells per 12 well were split on GelTrex coated 12 well plates. On DIV8 200 μl of a 30 mio cells/ml cell suspension were plated on 1.1 cm² of a poly-L-ornithine and laminin (PLO/lam) coated 6 well plate. During neural induction, rosettes should become visible. From DIV12, NI medium was changed to neural maintenance (NM) medium, with 20 ng/ml bFGF added within the first four days. On DIV22 rosettes were isolated with STEMdiff Neural Rosette Selection Reagent (STEMCELL Techn.) and plated on PLO/lam coated 6 well plates in NM medium containing bFGF. On DIV29 rosettes were split using accutase. On DIV39 NPCs were frozen in Neural progenitor freezing medium (STEMCELL Techn.).

For cortical neuron differentiation, NPCs were thawed in NM medium containing bFGF on PLO/lam coated 6 well plates. 1.5 mio NPCs were differentiated in maturation medium consisting of NB/B27 medium (Neurobasal medium, Penicillin-Streptomycin, 1×B27 supplement) together with additional factors (4 μM PD033291, 20 ng/ml BDNF, 20 ng/ml GDNF, 100 UM ascorbic acid, 0.5 mM CAMP, 1 μg/ml laminin) on a PLO/lam coated 6 well plate. After 1 week, 50000 cells were split into a 96 well plate using Accutase and ROCK inhibitor. Medium was changed half every 2 to 3 days. Neurons are differentiated for 2 weeks before coculturing with microglia. For coating, cell plating and medium changes the Integra ASSIST PLUS pipetting robot was used.

### Neuron-Microglia Coculture

2 days before adding the microglia to the neurons, PD0332991 was removed from the maturation medium. hiMGL on day 14 of the differentiation were used for the coculture. 8500 cells were added per 96 well to the neurons in NB/B27 medium with additional 25 ng/ml M-CSF, 100 ng/ml IL-34, and 50 ng/ml TGF-β1. Neurons and microglia were cocultured for 2 weeks before adding the Amyloid β.

### Amyloid β Treatment

Human amyloid β (1-42) (rPeptide, A-1170-02) or scrambled human Amyloid β (1-42) (rPeptide, A-1004-1) were incubated at 37° C. overnight for aggregation. Amyloid β was added to the coculture system at the indicated concentrations every 3 to 4 days by half medium change. The anti-TREM2 antibodies were added in parallel with Amyloid β for one to two weeks.

p-Syk AlphaLISA

Phosphorylated SYK (p-Syk) was measured using the AlphaLISA SureFire Ultra p-SYK Assay Kit (PerkinElmer, ALSU-PSYK-A-HV) following the manufacturer's instructions. Briefly, HEK293 cells overexpressing human TREM2 and human DAP12 were plated in 50 μl media at a density of 50,000 cells/well in a 96-well plate and incubated overnight at 37° C. in a cell culture incubator. The next day medium was removed and 50 μl of antibody diluted in medium was added to the cells. Following an incubation for 5 min at 37° C. treatment solutions were removed and cells were lysed with 50 μl lysis buffer supplemented with phosphatase inhibitor (VWR) for 10 min on a plate shaker (~350 rpm). 30 μl of lysate were then used for further incubation steps with acceptor and donor beads (each for 1 h) following analysis using a CLARIOstar^{Plus} Plate Reader (BMG Labtech).

For microglia cells, 96-well plates were pre-coated over night with antibody at 4° C. and microglia cells were added the following day at a density of 60,000 cells/well. Following an incubation for 10 min at 37° C. treatment solutions were removed and cells were lysed with 50 μl lysis buffer supplemented with phosphatase inhibitor (VWR) for 10 min on a plate shaker (~350 rpm). 30 μl of lysate were then used for further incubation steps with acceptor and donor beads (each for 1 h) following analysis using a CLARIOstar^{Plus} Plate Reader (BMG Labtech).

### ELISA-Based Binding Assay for Anti-TREM2 Antibodies

Anti-TREM2 antibody binding to the extracellular domain of TREM2 (ecTREM2) or to the human or mouse peptide fragment of the stalk region of TREM2 were quantified in an ELISA assay. All procedures were performed at room temperature and incubations were done on a microtiter plate shaker. ELISA plate was coated with 60 μl/well (final concentration 0.5 μg/ml) of the according ligand (ecTREM2 (Hölzel Diagnostika, 11084-H08H) or human or mouse TREM2 peptide fragment) in coating buffer (NaHCO₃) for 1 h. The coated plate was washed three times with PBS-T (PBS, 0.1% Tween-20), blocked with 100 μl/well of blocking solution (PBS-T, 3% milk powder) for 1 h, and washed again. Antibodies were pre-diluted 1:10 in PBS and set to a concentration of 1 μg/ml. A dilution series was performed with dilution steps of 1:3 and 1:10. 50 μl/well of diluted AB were transferred to the blocked ELISA plate and incubated for 1 h. The plate was washed three times with PBS-T and incubated with anti-human-Strep-POD (Jackson Immunoresearch, #109-035-098) diluted in PBS-T 1:10000 for 1 h. After washing three times, bound POD was detected by incubation with 100 μl/well of TMB substrate (Thermo Scientific, #34029) until a maximal optical density (OD) of about 1 to 2 was reached. Finally, the colorimetric reaction was stopped with 100 μl/well stopping solution (1M H₂SO₄) and the OD determined at a wavelength of 450 nm with a reference wavelength of 595 nm in a plate reader (SpectraMax i3xl).

### Immunocytochemistry (ICC) and Imaging

Cells were fixed in 4% paraformaldehyde for 20 min at room temperature and washed 3 times with PBS. For permeabilization and blocking, cells were incubated 1 hour at room temperature in 3% normal goat serum (Abcam), 0.3% TritonX-100 in PBS, followed by 3 times washing. The following primary antibodies were used for overnight incubation at 4° C.: Rabbit anti-synapsin 1 (1:500, Synptic Systems, #106 103), mouse anti-MAP2 (1:1500, Sigma Aldrich, #M9942). After 3 times washing, cells were incubated for 1 hour at room temperature with the following secondary antibodies: Donkey anti-Rabbit 488 (Thermo Fisher, #A32790), Donkey anti-mouse 647 (Thermo Fisher, #A32787). After 3 times washing with PBS, cells were stained with 10 μM DAPI. After 3 times washing with PBS, cells were imaged with the EVOS M7000 imaging system using a 20× objective. 13 pictures were taken per well with 6 wells per condition.

### CellProfiler Analysis

CellProfiler software (Carpenter et al., 2006) was applied for image analysis. Customized CellProfiler pipelines were generated for automated analysis of synapse numbers, total and dead nuclei counts and neurite areas. Binary images were applied to control for the correct segmentation of the images and the correct identification of the objects. In rare cases cell culture issues, e.g. pipetting errors, might cause outlier values for specific wells. Respective wells were checked by eye, to ensure a visible difference from average and outliers were statistically identified using the Grubbs' method (alpha=0.05). Identified outliers were then removed.

Neurite analysis: The MAP2 channel was used for neurite detection. To enhance the neurite structures, the tubeness enhancement method was applied. Neurites were detected using the Minimum Cross-entropy thresholding method. To consider cell density, neurite area was normalized to the total nuclei number per picture.

Nuclei analysis: The DAPI channel was used for nuclei detection. DAPI images were illumination corrected and nuclei were detected using the Otsu thresholding method. Dead nuclei were defined as the sum of apoptotic nuclei and small bright nuclei. Apoptotic nuclei were defined as minimum two small apoptotic bodies lying close to each other. To enhance the apoptotic bodies structures, the speckles enhancement method was applied. Apoptotic bodies were detected using the Otsu thresholding method. Small bright nuclei were filtered from nuclei based on intensity and area.

### Statistical Analysis

For comparison of more than two groups, one-way ANOVA was used. Statistical significance was set at *, p<0.05; **, p<0.01; and ***, p<0.001; and ****, p<0.0001.

### II. Results

### Example 1: Cloning and Analysis of Antibodies Directed Against hTREM2 Stalk Region

As described in methods, we identified several antibody clones which bound to the extracellular domain of TREM2 and to an epitope peptide derived from the human TREM2 stalk region with high affinity. These clones were structurally very related between each other, but not identical.

Based on an amino acid sequence comparison by BLAST, the heavy chain gene including a 19-amino acid (aa) leader peptide of clone H08 is 95% homologous to that of clone M07 (22 out of 471 amino acids differ-87% homology when considering only the variable regions of both antibody clones). The light chain gene including a 22-aa leader peptide of clone H08 is 88% homologous to M07 (27 out of 243 amino acids differ).

The heavy chain gene including a 19-aa leader peptide of clone M07 is 96% homologous to M05 (18 out of 471 amino acids differ-90% homology when considering only the variable regions of both antibody clones). The light chain gene including a 22-aa leader peptide of clone M07 is 85% homologous to M05 (40 out of 239 amino acids differ).

Similarly, the heavy chain gene including a 19-aa leader peptide of clone M07 is 96% homologous to M03 (18 out of 471 amino acids differ-90% homology when considering only the variable regions of both antibody clones). The light chain gene including a 22-aa leader peptide of clone M07 is 82% homologous to M03 (41 out of 239 amino acids differ).

### Example 2: Analysis of Antibody M07 Directed Against hTREM2 Stalk Region

All antibody clones including M07 were compared among each other and to an already established agonistic rat antibody directed against hTREM2 (H01) and a fully human control antibody (IgG1-LALA isotype). The rat anti-human TREM2 antibody H01 had been identified after immunization in rats and purified. This antibody binds to the extracellular domain of TREM2-no specific DNA sequence is available for this antibody.

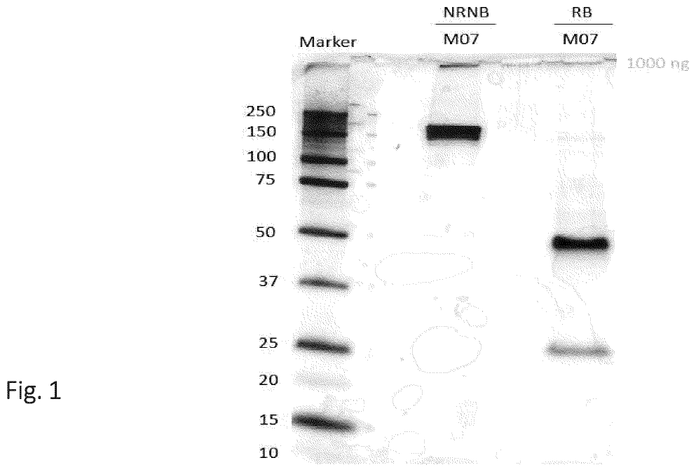

An SDS gel (silver staining) for M07 with reducing and non-reducing conditions is shown in FIG. 1. The antibody can be detected at ~150 kDa under non-reducing non-boiling (NRNB) conditions. Under reducing/boiling (RB) we detect the light chains of the antibody at 25 kDa and the heavy chains at 50 kDa (FIG. 1).

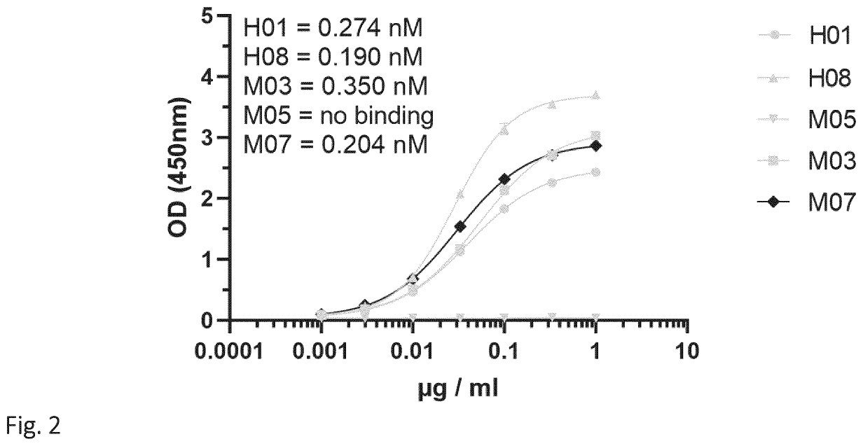

We determined the binding affinity of all antibody clones including M07 as well as of the control antibody H01 and negative control (not shown here), to ecTREM2 by an ELISA-based binding assay (FIG. 2). Whereas H08, M03 and M07 and the rat control antibody H01 showed affinities <350 pM to human ecTREM2 (190 pM, 350 pM 204 pM and 274 pM, respectively), antibody M05 did not bind to the ecTREM2 domain (FIG. 2).

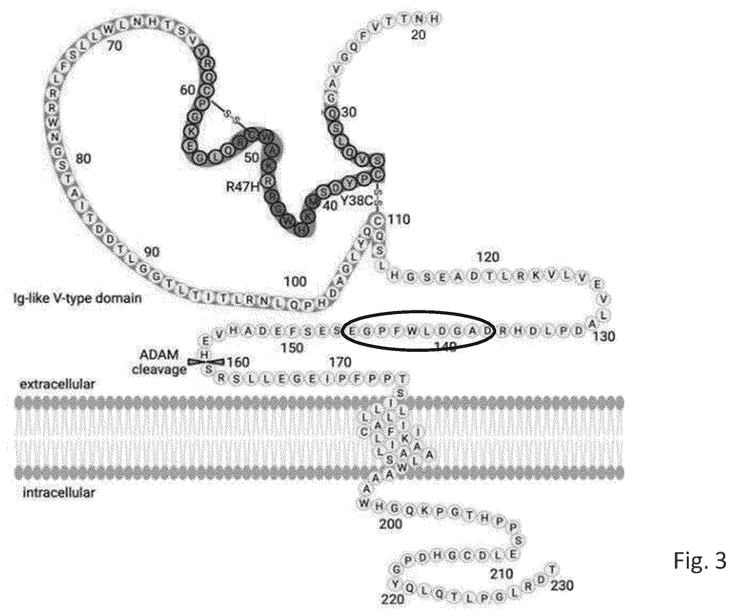

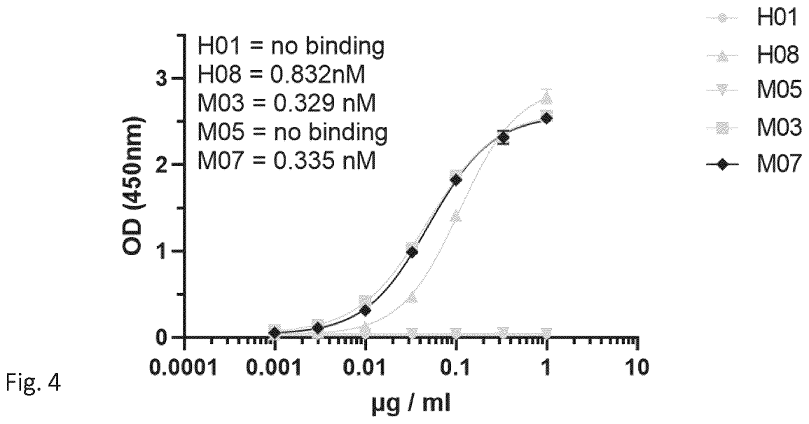

In a next step, we wanted to identify the exact antibody binding epitope of TREM2. We investigated a peptide fragment (DAGDLWFPG SEQ ID NO: 16) which represents a specific epitope from the human TREM2 stalk region (FIG. 3: oval) and performed the ELISA binding assay again.

Whereas antibodies H08, M03 and M07 showed affinities <850 pM to the specific epitope peptide (832 pM, 329 pM and 335 pM, respectively), M05 and the rat control antibody H01 did not bind to this epitope (FIG. 4).

### Example 3: Activation of TREM2 Signaling Using p-SYK Assay

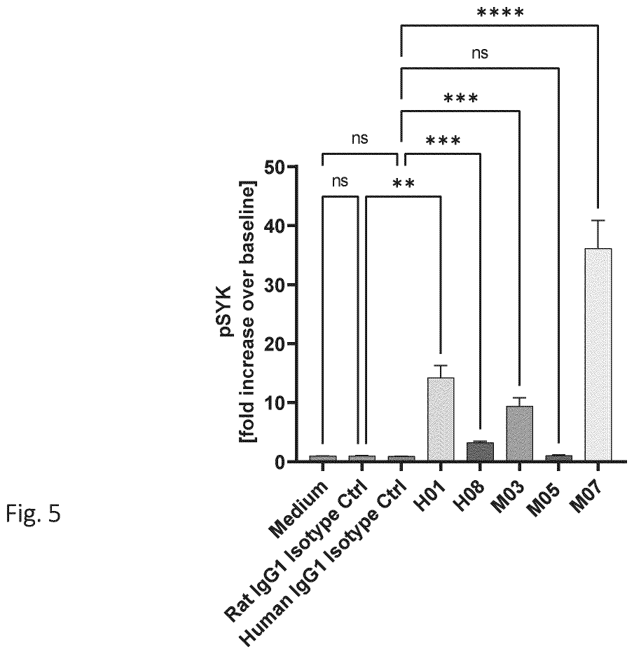

In order to test activation of TREM2 signaling via anti-TREM2 antibodies we employed a p-SYK AlphaLISA assay, which allows detection of human pSYK phosphorylation that occurs upon ligand binding to human TREM2. In order to analyze the activation of the TREM2 pathway, HEK293 cells stably expressing human TREM2 and human DAP12 were incubated with medium or with medium containing the various antibodies at a concentration of 40 μg/ml for 5 min at 37° C. and SYK phosphorylation was determined thereafter. The appropriate controls (human IgG1-LALA and rat IgG isotype antibodies) were also used in this assay. Treatment of HEK293-Flp-In hTREM2/hDAP12 cells with M07 results in strongly increased p-SYK levels compared to baseline control (isotype or medium only). M07 elicits an average 36-fold activation over baseline and hence activates pSYK much more potently than H01 or M03 (FIG. 5), whereas H08 induced very little, and M05 did not elicit any activation at all. The increase over baseline is shown in the following table.

| | H01 | H08 | M03 | M05 | M07 |
|---|---|---|---|---|---|
| Mean values of fold | 14.25 | 3.22 | 9.39 | 1.07 | 36.15 |
| increase over baseline | | | | | |

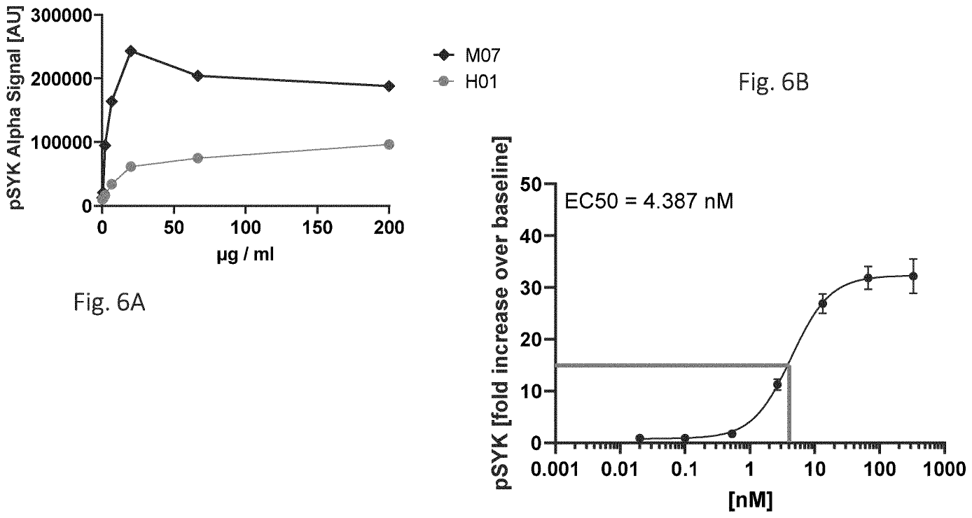

We also performed a titration series of the best activating antibody clones (H01 and M07) (FIG. 6).

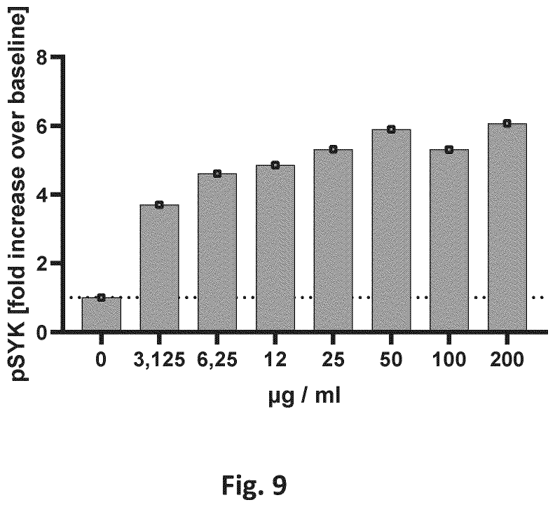

In order to test activation of TREM2 signaling in a cell type that endogenously expresses the TREM2 receptor, we differentiated microglia cells from hiPSC. The pSYK AlphaLISA assay described before was used to determine pSYK phosphorylation in hiPSC-derived microglia cells, using increasing concentrations of M07 antibody. Treatment of microglia cells with M07 results in a strong, concentration dependent increase in p-SYK levels (FIG. 9).

### Example 4: Induction of Neurodegeneration in Alzheimer's Disease Model

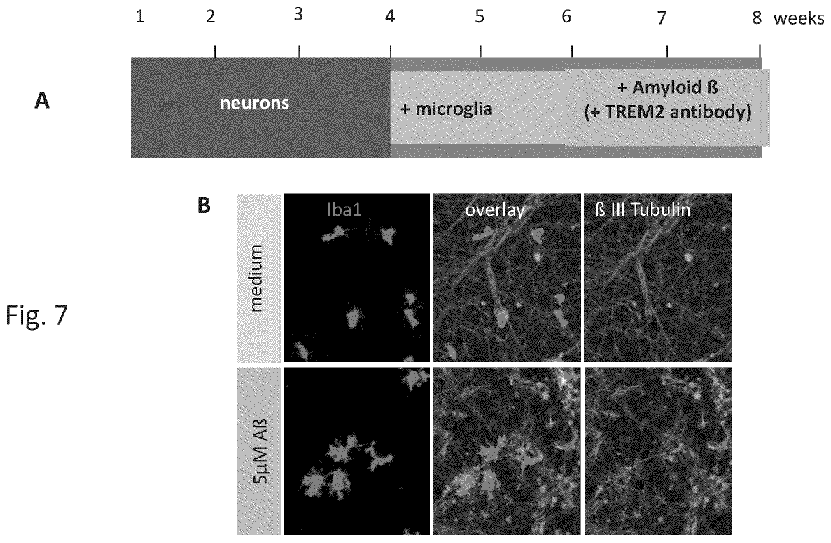

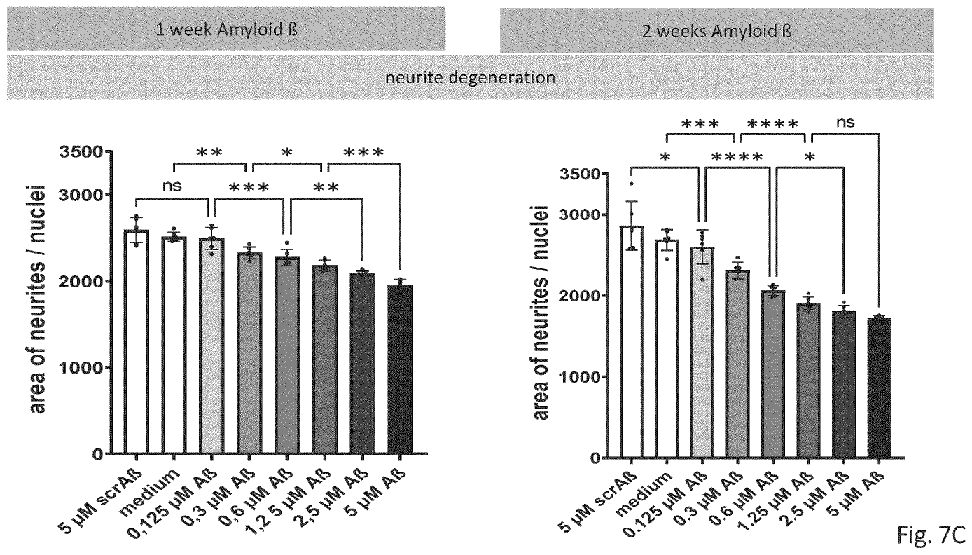

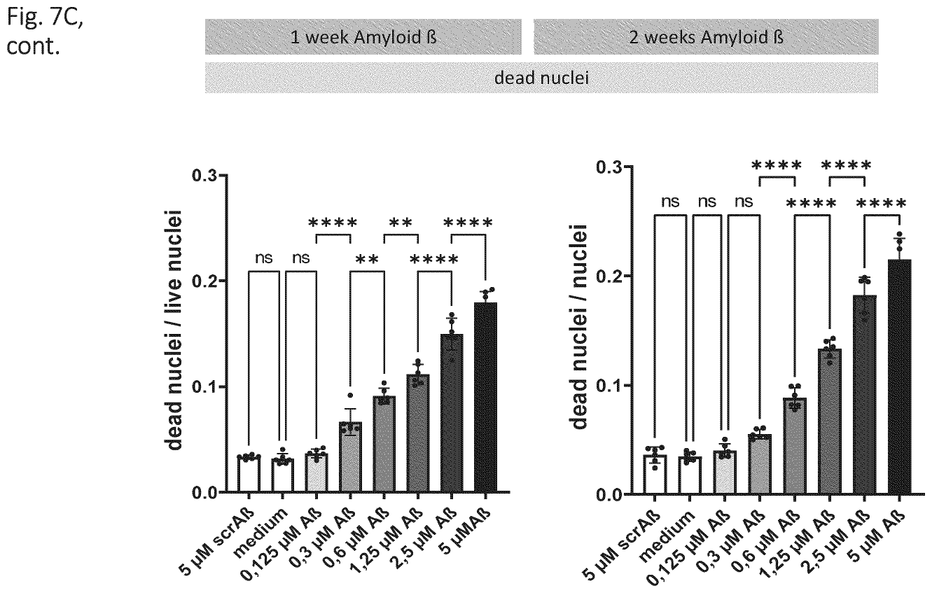

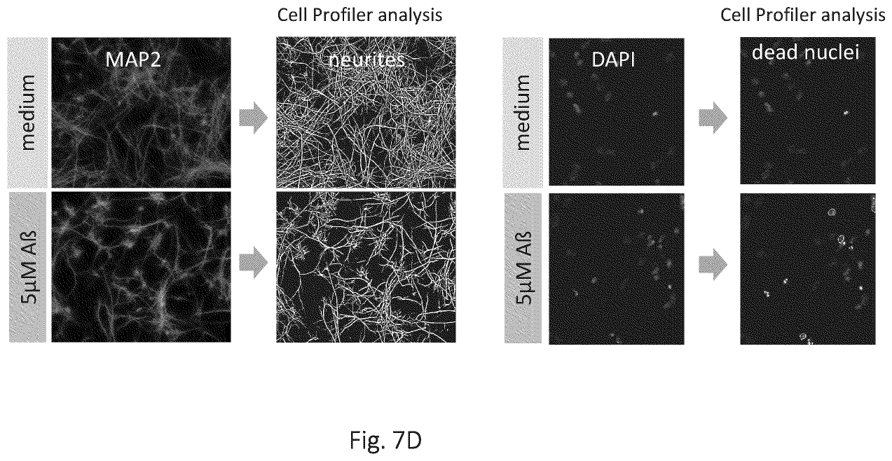

Amyloid β was added to a microglia neuron coculture system to induce neurodegeneration as a model for Alzheimer's disease. Microglia showed the expected ramified morphology in control conditions with only medium and an amoeboid morphology with addition of amyloid β (FIG. 7B). An amyloid β dose-dependent effect on neurodegeneration was observed. Neurite degeneration was characteristic for neurodegeneration and was measured by MAP2⁺ neurite area/live nuclei. Neuronal death, another hallmark of neurodegeneration was quantified by detecting dead nuclei numbers/total nuclei (FIG. 7C, D). This neurodegeneration model was used to determine a neuroprotective effect of the anti-hTREM2 antibody M07 on neurons.

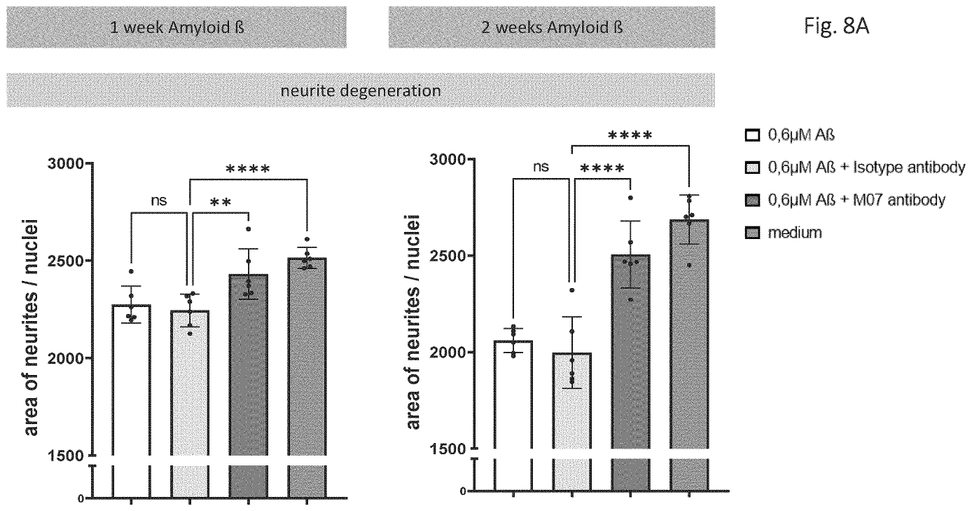

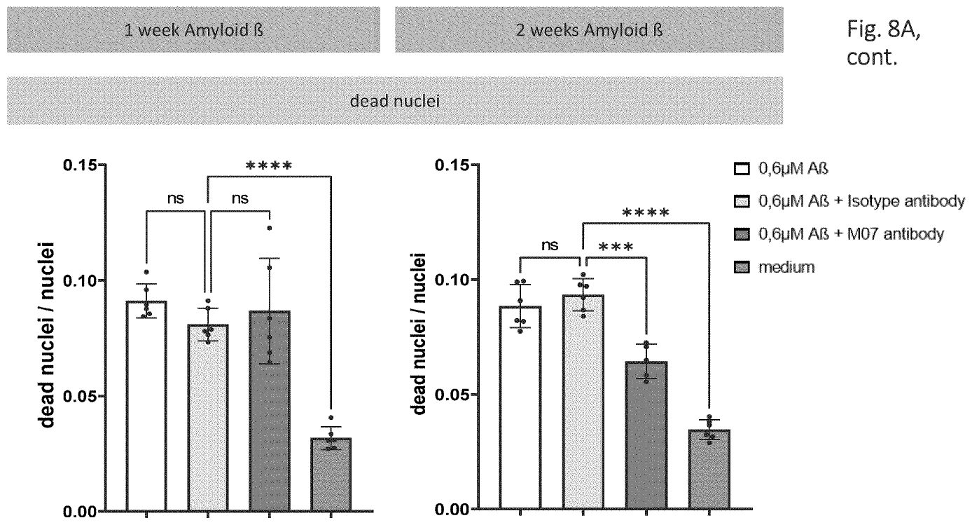

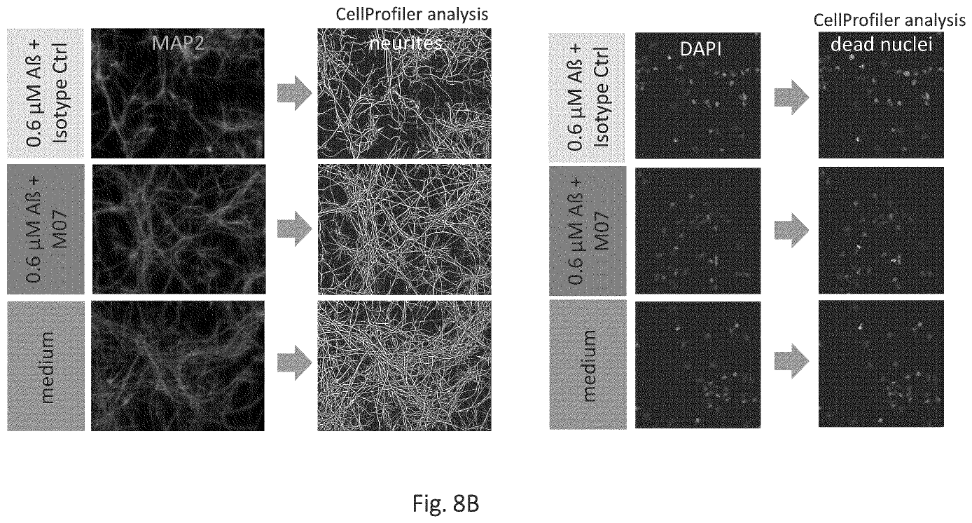

FIG. 8 shows that amyloid β-dependent neurodegeneration was significantly reduced by adding the anti-hTREM2 antibody M07 as compared to the isotype antibody. In detail, the M07 antibody reduced neurite degeneration at 1 week and 2 weeks of antibody addition. For the number of dead cells per nuclei, the M07 antibody reduced dead nuclei count at 2 weeks of antibody addition.

### Example 5: HTREM2 Antibody Reduces Neurodegeneration in a hiPSC-Derived Microglia-Neuron Co-Culture Model of Alzheimer's Disease

Amyloid β was added to a microglia neuron coculture system to induce neurodegeneration as a model for Alzheimer's disease (FIG. 7A). Microglia show the expected ramified morphology in control conditions with only medium and an amoeboid morphology with addition of amyloid β (FIG. 7B). An amyloid β dose-dependent effect on neurodegeneration can be measured. Neurite degeneration is characteristic for neurodegeneration and was measured by MAP2+ neurite area/live nuclei. Neuronal death, another hallmark of neurodegeneration was quantified detecting dead nuclei numbers/total nuclei (FIG. 7C+D). This neurodegeneration model was used to determine a neuroprotective effect of the anti-hTREM2 antibody M07 on neurons.

FIG. 8 shows that amyloid β-dependent neurodegeneration was significantly reduced by adding the anti-hTREM2 antibody M07 as compared to the isotype antibody. In detail, the M07 antibody reduced neurite degeneration at 1 week and 2 weeks of antibody addition. For the number of dead cells per nuclei, the M07 antibody reduced dead nuclei count at 2 weeks of antibody addition.

### III. Discussion and Conclusion

Using phage display technology, we have obtained fully human anti-TREM2 antibodies which were initially screened for antigen binding. The selected fully human IgG1-LALA modified antibodies were employed to determine the binding affinity to the extracellular domain of human TREM2, activation of human TREM2 signaling (human TREM2/DAP-dependent SYK phosphorylation), and most importantly, efficacy in a complex, relevant AD model using human brain cells which were differentiated from human induced pluripotent stem cells (hiPSC).

The LALA-modification strongly reduces effector function of IgG1 antibodies, which is important for studies with human immune and neuronal cells. The fully human backbones of the antibodies which we have generated are advantageous compared to existing humanized antibodies which are based on identification of clones in non-human animal immune systems (e.g., US2017240631A1-Alector AL-002, and WO2020172450 A1-Denali), because less immunological complications can be expected using fully human antibodies upon repeated preventive or therapeutic applications in vivo in humans.

Initially, we identified a variety of structurally similar and related antibodies (heavy chain amino acid sequence homology 90% or more) which all bound to the extracellular domain of human TREM2 with high affinities (below 10-9 M). Much to our surprise, we then found that only one of these antibodies could strongly induce human TREM2/DAP-triggered SYK phosphorylation, which is the pivotal TREM2-dependent effector pathway in AD. SYK phosphorylation was up to 60-fold increased by M07, whereas no or little activation was seen with the other antibody clones, although M07 binds to the same epitope on the extracellular domain of TREM2 as H08 and M03.

The increase in human TREM2/DAP-dependent pSYK level that was induced by M07 was much stronger than that observed for published as well as patented agonistic antibodies against human TREM2, and especially of any human anti-human TREM2 antibodies. The hT2AB antibody disclosed by AMGEN (WO2022120373 A1) was used in pSYK assays comparable to ours and caused a 12-fold increase over baseline (Ellwanger et al., 2021). Alector presented in its patent application (US2017240631 A1) numerous antibodies directed against human anti-TREM2. Phosphorylation of SYK was shown on protein level and a ~3-4 fold increase was reported for antibodies #22, #45 and #65 in human dendritic cells. In human macrophages, a 6-fold increase in SYK phosphorylation was observed. Denali presented several anti-hTREM2 antibodies in their patent application (WO2020172450 A1)-one of which is CL0020188. This antibody increased pSYK levels 4-fold compared to control antibody in TREM2-expressing HEK293 cells. Data from Fassler et al. showed pSYK activation on protein level mediated by an anti-hTREM2 antibody, however without quantification of the increase (Fassler et al., 2021).

In addition, the inventors have used an innovative complex and relevant AD model using human brain cells which were differentiated from human induced pluripotent stem cells (hiPSC). None of the anti-TREM2 antibodies, which had been known in the state of the art, had been analyzed in a comparably sophisticated AD model using hiPSC-derived neurons and microglia. For instance, WO2020172450 A1 (Denali) disclosed a phagocytosis assay using hiPSC-derived microglia and amyloid β. However, the analysis was not performed in coculture with neurons. Therefore, the benefit of amyloid β phagocytosis on neurons cannot be determined.

AD can also be studied in other disease models which all have inherent limitations. Most researchers still work in mouse models, which however often failed to predict clinical efficacy of anti-AD drug candidates.

The present invention solves the problem identified above and surprisingly provides fully human anti-human TREM2 antibodies which activate human TREM2-dependent human pSYK signaling so strongly that beneficial effects can be observed in a relevant AD model of human brain cells. Previous strong activators were only described for mouse TREM2 in mouse cells.

### CITED REFERENCES

Abud, E. M., Ramirez, R. N., Martinez, E. S., Healy, L. M., Nguyen, C. H. H., Newman, S. A., Yeromin, A. V., Scarfone, V. M., Marsh, S. E., Fimbres, C., Caraway, C. A., Fote, G. M., Madany, A. M., Agrawal, A., Kayed, R., Gylys, K. H., Cahalan, M. D., Cummings, B. J., Antel, J. P., . . . Blurton-Jones, M. (2017). iPSC-Derived Human Microglia-like Cells to Study Neurological Diseases. Neuron, 94 (2), 278-293.e9. https://doi.org/10.1016/j.neuron.2017.03.042
Carpenter, A. E., Jones, T. R., Lamprecht, M. R., Clarke, C., Kang, I. H., Friman, O., Guertin, D. A., Chang, J. H., Lindquist, R. A., Moffat, J., Golland, P., & Sabatini, D. M. (2006). CellProfiler: Image analysis software for identifying and quantifying cell phenotypes. Genome Biology, 7 (10). https://doi.org/10.1186/gb-2006-7-10-r100
Ellwanger, D. C., Wang, S., Brioschi, S., Shao, Z., Green, L., Case, R., Yoo, D., Weishuhn, D., Rathanaswami, P., Bradley, J., Rao, S., Cha, D., Luan, P., Sambashivan, S., Gilfillan, S., Hasson, S. A., Foltz, I. N., van Lookeren Campagne, M., & Colonna, M. (2021). Prior activation state shapes the microglia response to antihuman TREM2 in a mouse model of Alzheimer's disease. Proceedings of the National Academy of Sciences of the United States of America, 118 (3), 1-12. https://doi.org/10.1073/PNAS.2017742118
English, H., Hong, J., & Ho, M. (2020). Ancient species offers contemporary therapeutics: An update on shark VNAR single domain antibody sequences, phage libraries and potential clinical applications. Antibody Therapeutics, 3 (1), 1-9. https://doi.org/10.1093/ABT/TBAA001
Fassler, M., Rappaport, M. S., Cuño, C. B., & George, J. (2021). Engagement of TREM2 by a novel monoclonal antibody induces activation of microglia and improves cognitive function in Alzheimer's disease models. Journal of Neuroinflammation, 18 (1), 1-18. https://doi.org/10.1186/s12974-020-01980-5
Gregg, A., Teo, S., Tessier-Lavigne, M., Jacob, S., Olsen, K. M., Noggle, S., Paquet, D., Kwart, D., Chen, A., & Sproul, A. (2016). Efficient introduction of specific homozygous and heterozygous mutations using CRISPR/Cas9. Nature, 533 (7601), 125-129. https://doi.org/10.1038/nature17664
Harmsen, M. M., & De Haard, H. J. (2007). Properties, production, and applications of camelid single-domain antibody fragments. Applied Microbiology and Biotechnology, 77 (1), 13-22. https://doi.org/10.1007/s00253-007-1142-2
Keren-Shaul, H., Spinrad, A., Weiner, A., Matcovitch-Natan, O., Dvir-Szternfeld, R., Ulland, T. K., David, E., Baruch, K., Lara-Astaiso, D., Toth, B., Itzkovitz, S., Colonna, M., Schwartz, M., & Amit, I. (2017). A Unique Microglia Type Associated with Restricting Development of Alzheimer's Disease. Cell, 169 (7), 1276-1290.e17. https://doi.org/10.1016/j.cell.2017.05.018
Kleinberger, G, Yamanishi, Y., Suárez-Calvet, M., Czirr, E., Lohmann, E., Cuyvers, E., Struyfs, H., Pettkus, N., Wenninger-Weinzierl, A., Mazaheri, F., Tahirovic, S., Lleó, A., Alcolea, D., Fortea, J., Willem, M., Lammich, S., Molinuevo, J., Sánchez-Valle, R., Antonell, A., . . . Haass, C. (2014). TREM2 mutations implicated in neurodegeneration impair cell surface transport and phagocytosis. Science Translational Medicine, 6(243). https://doi.org/10.1126/scitranslmed.3009093
Kleinberger, Gernot, Brendel, M., Mracsko, E., Wefers, B., Groeneweg, L., Xiang, X., Focke, C., Deußing, M., Suárez-Calvet, M., Mazaheri, F., Parhizkar, S., Pettkus, N., Wurst, W., Feederle, R., Bartenstein, P., Mueggler, T., Arzberger, T., Knuesel, I., Rominger, A., & Haass, C. (2017). The FTD-like syndrome causing TREM2 T66M mutation impairs microglia function, brain perfusion, and glucose metabolism. The EMBO Journal, 36 (13), 1837-1853. https://doi.org/10.15252/embj.201796516
McQuade, A., Coburn, M., Tu, C. H., Hasselmann, J., Davtyan, H., & Blurton-Jones, M. (2018). Development and validation of a simplified method to generate human microglia from pluripotent stem cells. Molecular Neurodegeneration, 13 (1), 67. https://doi.org/10.1186/s13024-018-0297-x
Reifschneider, A., Robinson, S., van Lengerich, B., Gnorich, J., Logan, T., Heindl, S., Vogt, M. A., Weidinger, E., Riedl, L., Wind, K., Zatcepin, A., Pesamaa, I., Haberl, S., Nuscher, B., Kleinberger, G., Klimmt, J., Götzl, J. K., Liesz, A., Bürger, K., . . . Haass, C. (2022). Loss of TREM2 rescues hyperactivation of microglia, but not lysosomal deficits and neurotoxicity in models of progranulin deficiency. The EMBO Journal, 41 (4), 1-25. https://doi.org/10.15252/embj.2021109108
Schlepckow, K., Kleinberger, G., Fukumori, A., Feederle, R., Lichtenthaler, S. F., Steiner, H., & Haass, C. (2017). An Alzheimer-associated TREM2 variant occurs at the ADAM cleavage site and affects shedding and phagocytic function. EMBO Molecular Medicine, 9 (10), 1356-1365. https://doi.org/10.15252/emmm.201707672
Song, W., Hooli, B., Mullin, K., Jin, S. C., Cella, M., Ulland, T. K., Wang, Y., Tanzi, R. E., & Colonna, M. (2017). Alzheimer's disease-associated TREM2 variants exhibit either decreased or increased ligand-dependent activation. Alzheimer's and Dementia, 13 (4), 381-387. https://doi.org/10.1016/j.jalz.2016.07.004
Suárez-Calvet, M., Caballero, M. A. A., Kleinberger, G., Bateman, R. J., Fagan, A. M., Morris, J. C., Levin, J., Danek, A., Ewers, M., & Haass, C. (2016). Early changes in CSF sTREM2 in dominantly inherited Alzheimer's disease occur after amyloid deposition and neuronal injury. Science Translational Medicine, 8 (369), 34-38. https://doi.org/10.1126/scitranslmed.aag1767
Suárez-Calvet, M., Kleinberger, G., Araque Caballero, M. A., Brendel, M., Rominger, A., Alcolea, D., Fortea, J., Lleó, A., Blesa, R., Gispert, J. D., Sánchez-Valle, R., Antonell, A., Rami, L., Molinuevo, J. L., Brosseron, F., Traschutz, A., Heneka, M. T., Struyfs, H., Engelborghs, S., . . . Haass, C. (2016). sTREM2 cerebrospinal fluid levels are a potential biomarker for microglia activity in early-stage Alzheimer's disease and associate with neuronal injury markers. EMBO Molecular Medicine, 8 (5), 466-476. https://doi.org/10.15252/emmm.201506123
Ulland, T. K., Song, W. M., Huang, S. C. C., Ulrich, J. D., Sergushichev, A., Beatty, W. L., Loboda, A. A., Zhou, Y., Cairns, N. J., Kambal, A., Loginicheva, E., Gilfillan, S., Cella, M., Virgin, H. W., Unanue, E. R., Wang, Y., Artyomov, M. N., Holtzman, D. M., & Colonna, M. (2017). TREM2 Maintains Microglial Metabolic Fitness in Alzheimer's Disease. Cell, 170 (4), 649-663.e13. https://doi.org/10.1016/j.cell.2017.07.023
Wunderlich, P., Glebov, K., Kemmerling, N., Tien, N. T., Neumann, H., & Walter, J. (2013). Sequential proteolytic processing of the triggering receptor expressed on myeloid cells-2 (TREM2) protein by ectodomain shedding and γ-secretase-dependent intramembranous cleavage. Journal of Biological Chemistry, 288 (46), 33027-33036. https://doi.org/10.1074/jbc.M113.517540
Zhong, L., Chen, X.-F., Wang, T., Wang, Z., Liao, C., Wang, Z., Huang, R., Wang, D., Li, X., Wu, L., Jia, L., Zheng, H., Painter, M., Atagi, Y., Liu, C.-C., Zhang, Y.-W., Fryer, J. D., Xu, H., & Bu, G. (2017). Soluble TREM2 induces inflammatory responses and enhances microglial survival. The Journal of Experimental Medicine, jem.20160844. https://doi.org/10.1084/jem.20160844
Zhong, L., & Chen, X. F. (2019). The Emerging Roles and Therapeutic Potential of Soluble TREM2 in Alzheimer's Disease. Frontiers in Aging Neuroscience, 11 (November), 1-9. https://doi.org/10.3389/fnagi.2019.00328
Zhong, L., Wang, T., & Zhuo, R. (2019). Soluble TREM2 ameliorates pathological phenotypes by modulating microglial functions in an Alzheimer's disease model. Nature Communications, 904, 1-16. https://doi.org/10.1038/s41467-019-09118-9

### Nucleotide and Amino Acid Sequences

SEQ ID NO: 1 Variable region or domain of the light chain of clone M07 (107 aa):
DIQLTQSPLSLSASAGDRVTITCRASQSIRDYLGWYQQKPGKAPKLLIYAASKLQSGV
PSRFSGSGSGTDFTLTISSLQPEDFATYYCQQSYHTPPFTFGQGTKVEI
SEQ ID NO: 2 Variable region or domain of heavy chain of clone M07 (122 aa):
EVQLLESGGGLVQPGGSLRLTCAASGFTFSSYAMSWVRQAPGKGLEWVSVINGRGSNTYYADSVKGR
FTITRDNSKNTLYLEMNSLRAEDTAVYYCARVRAYSGPSYGFDYWGQGTLVTVSS
SEQ ID NO: 3 CDR-L1 of M07: QSIRDY
SEQ ID NO: 4 CDR-L2 of M07: AAS
SEQ ID NO: 5 CDR-L3 of M07: QQSYHTPPFT
SEQ ID NO: 6 CDR-H1 of M07: GFTFSSYA
SEQ ID NO: 7 CDR-H2 of M07: NGRGSNT
SEQ ID NO: 8 CDR-H3 of M07: ARVRAYSGPSYGFDY
SEQ ID NO: 9 Kappa-1 light chain of M07 with 22 amino acid residue N-terminal leader
(237 aa), sequences of/in supposed CDRs 1 to 3 underlined:
MDMRVPAQLLGLLLLWLSGARCDIQLTQSPLSLSASAGDRVTITCRASQSIRDYLGWYQQKPGKAPK
LLIYAASKLQSGVPSRFSGSGSGTDFTLTISSLQPEDFATYYCQQSYHTPPFTFGQGTKVEIKRTVA
APSVEIFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 10 Kappa-1 light chain of M07 (215 aa), sequences of/in supposed CDRs 1 to
3 underlined:
DIQLTQSPLSLSASAGDRVTITCRASQSIRDYLGWYQQKPGKAPKLLIYAASKLQSGVPSRFSGSGS
GTDFTLTISSLQPEDFATYYCQQSYHTPPFTFGQGTKVEIKRTVAAPSVFIFPPSDEQLKSGTASVV
CLLNNFYPREAKVQWKVDNALQSGNSQESVTEQDSKDSTYSLSSTLTLSKADYEKHKVYACEVTHQG
LSSPVTKSFNRGEC
SEQ ID NO: 11 Lambda light chain of M07 (213 aa), sequences of/in supposed CDRs 1 to
3 underlined:
DIQLTQSPLSLSASAGDRVTITCRASQSIRDYLGWYQQKPGKAPKLLIYAASKLQSGVPSRESGSGS
GTDFTLTISSLQPEDFATYYCQQSYHTPPFTFGQGTKVEIKGQPKAAPSVTLFPPSSEELQANKATL
VCLISDFYPGAVTVAWKADSSPVKAGVETTTPSKQSNNKYAASSYLSLTPEQWKSHRSYSCQVTHEG
STVEKTVAPTEC
SEQ ID NO: 12 Heavy chain full IgG1-LALA-TFN (including a binding segment for human
transferrin receptor hTfR in CH3 domain) of clone M07 with N-terminal leader (471 aa),
sequences of/in supposed CDRs 1 to 3 underlined:
MELGLSWIFLLAILKGVQCEVQLLESGGGLVQPGGSLRLTCAASGFTFSSYAMSWVRQAPGKGLEWVSVINGRG
SNTYYADSVKGRFTITRDNSKNTLYLEMNSLRAEDTAVYYCARVRAYSGPSYGFDYWGQGTLVTVSSASTKGPS
VFPLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESYGTEWSSYKTTPPVLDSDGSFFLYSKLTVTKSEWQQG
FVFSCSVMHEALHNHYTQKSLSLSPGK
SEQ ID NO: 13 Heavy chain full IgG1-LALA-TFN (452 aa) of clone M07, sequences of/in
supposed CDRs 1 to 3 underlined:
EVQLLESGGGLVQPGGSLRLTCAASGFTFSSYAMSWVRQAPGKGLEWVSVINGRGSNTYYADSVKGRFTITRDN
SKNTLYLEMNSLRAEDTAVYYCARVRAYSGPSYGFDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELTKNQVSL
TCLVKGFYPSDIAVEWESYGTEWSSYKTTPPVLDSDGSFFLYSKLTVTKSEWQQGFVFSCSVMHEALHNHYTQK
SLSLSPGK
SEQ ID NO: 14 Heavy chain full IgG1-LALA (471 aa) with N-terminal leader, sequences
of/in supposed CDRs 1 to 3 underlined:
MELGLSWIFLLAILKGVQCEVQLLESGGGLVQPGGSLRLTCAASGFTFSSYAMSWVRQAPGKGLEWVSVINGRG
SNTYYADSVKGRFTITRDNSKNTLYLEMNSLRAEDTAVYYCARVRAYSGPSYGFDYWGQGTLVTVSSASTKGPS
VFPLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKSRWQQG
NVFSCSVMHEALHNHYTQKSLSLSPGK
SEQ ID NO: 15 Heavy chain full IgG1-LALA (452 aa), sequences of/in supposed CDRs 1 to
3 underlined:
EVQLLESGGGLVQPGGSLRLTCAASGFTFSSYAMSWVRQAPGKGLEWVSVINGRGSNTYYADSVKGRFTITRDN
SKNTLYLEMNSLRAEDTAVYYCARVRAYSGPSYGFDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHEALHNHYTQK
SLSLSPGK
SEQ ID NO: 16: peptide fragment representing epitope from the hTREM2 stalk region:
DAGDLWFPG
SEQ ID NO: 17 Kappa-1 light chain of H08, sequences of/in supposed CDRs 1 to 3
underlined:
AIRMTQSPDSLPVSLGERATINCKSSQSVLYGSNNKNYLAWYQQKPGQPPKLLIYWASTRESGVPDRFSGSGSG
TDFTLTISSLQAEDVAVYYCQQYYSTPLTFGPGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSENRGEC
SEQ ID NO: 18 Heavy chain full IgG1-LALA of clone H08, sequences of/in supposed CDRs
1 to 3 underlined:
EVQLVQSGGGLVQPGGSLRLSCAASGFTFSSYAMSWVRQAPGKGLEWVSAISGSGGSTYYADSVKGRFTISRDN
AKRSLYLQMNDLRVEDTAVYYCARTRTNVFDFWGQGTMVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEALHNHYTQKSLSLS
PGK
SEQ ID NO: 19 Kappa-1 Light chain of full IgG1 of M03, sequences of/in supposed CDRs 1
to 3 underlined:
DIRLTQPPSVSGAPGQRVTISCSGSSSNIGSLFVSWYQQLPGTAPKLLIYSNSQHPSGVPDRFSGSKSGTSASL
AITGLQAEDEADYYCSAYDQFSNSVVFGGGTKLTVLRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEC
SEQ ID NO: 20 Lambda Light chain of full IgG1 of M03, sequences of/in supposed CDRs 1
to 3 underlined:
DIRLTQPPSVSGAPGQRVTISCSGSSSNIGSLFVSWYQQLPGTAPKLLIYSNSQHPSGVPDRFSGSKSGTSASL
AITGLQAEDEADYYCSAYDQFSNSVVFGGGTKLTVLGQPKAAPSVTLFPPSSEELQANKATLVCLISDFYPGAV
TVAWKADSSPVKAGVETTTPSKQSNNKYAASSYLSLTPEQWKSHRSYSCQVTHEGSTVEKTVAPTEA
SEQ ID NO: 21 Heavy chain full IgG1-LALA of clone M03; sequences of/in supposed CDRs
1 to 3 underlined:
EVQLVQSGGGLVQPGGSLRLSCAASGFTFSSYGITWVRQAPGKGLEWVSFISGGGSYTYYADSVKGRFTISRDN
AKRTLYLQMNSLRAEDTAVYYCARSGRAYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEALHNHYTQKSLSLS
PGKYTEWSS
SEQ ID NO: 22 Kappa-1 Light chain of full IgG1 of M05, sequences of/in supposed CDRs 1
to 3 underlined:
DIVMTQPPSVSVTPGQRVTISCRSSSSNIGSLFVSWYQQLPGTAPKLLIYSNSQHPSGVPDRFSGSKSGTSASL
AITGLQAEDEADYYCSAYDQFSNSVVFGGGTKLTVLRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSFNRGEA
SEQ ID NO: 23 Lambda Light chain of full IgG1 of M05, sequences of/in supposed CDRs 1
to 3 underlined:
DIVMTQPPSVSVTPGQRVTISCRSSSSNIGSLFVSWYQQLPGTAPKLLIYSNSQHPSGVPDRFSGSKSGTSASL
AITGLQAEDEADYYCSAYDQFSNSVVFGGGTKLTVLGQPKAAPSVTLFPPSSEELQANKATLVCLISDFYPGAV
TVAWKADSSPVKAGVETTTPSKQSNNKYAASSYLSLTPEQWKSHRSYSCQVTHEGSTVEKTVAPTEA
SEQ ID NO: 24 Heavy chain full IgG1-LALA of clone M05, sequences of/in supposed CDRs
1 to 3 underlined:
EVQLLESGGGLVQPGGSLRLSCAASGFTFSSNAMTWVRQAPGKGLEWVSVIGSSGSYTYYADSVKGRFTISRDN
SKNTLYLQMNSLRAEDTAVYYCARSGTTGFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQGNVFSCSVMHEALHNHYTQKSLSLS
PGKYTEWSS
SEQ ID NO: 25 Coding sequence of kappa-1 light chain of antibody M07 with N-terminal
leader (723 bp):
CATCATGGACATGAGAGTGCCCGCTCAGCTGCTGGGACTGCTGTTGTTGTGGCTGTCTGGCGCTAGATGCGACA
TCCAGCTGACCCAGTCTCCACTGTCTCTGTCTGCCTCTGCTGGCGACAGAGTGACCATCACCTGTCGGGCCTCT
CAGTCTATCAGAGACTACCTCGGCTGGTATCAGCAGAAGCCTGGCAAGGCTCCCAAGCTGCTGATCTACGCTGC
CTCTAAACTGCAGTCCGGCGTGCCCTCTAGATTCTCTGGCTCTGGATCTGGCACCGACTTCACCCTGACCATCA
GTTCTCTGCAGCCTGAGGACTTCGCCACCTACTACTGCCAGCAGTCCTATCACACCCCTCCATTCACCTTTGGC
CAGGGCACCAAGGTGGAAATCAAGAGAACCGTGGCCGCTCCTTCCGTGTTCATCTTCCCACCATCTGACGAGCA
GCTGAAGTCCGGCACAGCTTCTGTCGTGTGCCTGCTGAACAACTTCTACCCTCGGGAAGCCAAGGTGCAGTGGA
AGGTGGACAATGCCCTGCAGTCTGGCAACTCCCAAGAGTCTGTGACCGAGCAGGACTCCAAGGACTCTACCTAC
AGCCTGTCCTCCACACTGACCCTGTCTAAGGCCGACTACGAGAAGCACAAGGTGTACGCCTGTGAAGTGACCCA
CCAGGGACTGTCTAGCCCCGTGACCAAGTCTTTCAACAGAGGCGAGTGCTGATTAAT
SEQ ID NO: 26 Coding sequence of heavy chain of antibody M07 with N-terminal leader
(1424 bp):
CTAGGATGGAACTGGGCCTGTCCTGGATCTTTCTGCTGGCTATTCTGAAGGGCGTGCAGTGCGAAGTGCAGCTG
TTGGAATCTGGCGGAGGATTGGTTCAGCCTGGCGGCTCTCTGAGACTGACCTGTGCTGCCTCTGGCTTCACCTT
CTCCTCTTACGCCATGTCCTGGGTCCGACAGGCTCCTGGAAAAGGACTGGAATGGGTGTCCGTGATCAACGGCA
GAGGCTCCAACACCTACTACGCCGACTCTGTGAAGGGCAGATTCACCATCACCAGAGACAACTCCAAGAACACC
CTGTACCTGGAAATGAACTCCCTGAGAGCCGAGGACACCGCCGTGTACTACTGTGCTAGAGTGCGGGCTTACTC
TGGCCCTTCCTACGGCTTTGATTATTGGGGCCAGGGCACCCTGGTCACCGTGTCCTCTGCTTCTACAAAGGGCC
CCTCTGTGTTCCCTCTGGCTCCTAGCTCTAAGTCCACCTCTGGTGGAACCGCTGCTCTGGGCTGTCTGGTCAAG
GATTACTTCCCTGAGCCTGTGACCGTGTCTTGGAATTCCGGTGCTCTGACCTCCGGCGTGCACACATTTCCAGC
TGTGCTGCAGTCCTCCGGCCTGTACTCTCTGTCCTCTGTCGTGACCGTGCCTTCTAGCTCTCTGGGCACCCAGA
CCTACATCTGCAACGTGAACCACAAGCCTTCCAACACCAAAGTGGACAAGAAGGTGGAACCCAAGTCCTGCGAC
AAGACCCACACCTGTCCACCTTGTCCTGCTCCAGAAGCTGCTGGCGGACCCTCCGTTTTCCTGTTTCCACCTAA
GCCTAAGGACACCCTGATGATCTCTCGGACCCCTGAAGTGACCTGCGTGGTGGTGGATGTGTCTCACGAGGATC
CCGAAGTGAAGTTCAATTGGTACGTGGACGGCGTGGAAGTGCACAACGCCAAGACCAAGCCTAGAGAGGAACAG
TACAACTCCACCTACAGAGTGGTGTCTGTGCTGACAGTGCTGCACCAGGACTGGCTGAACGGCAAAGAGTACAA
GTGCAAGGTGTCCAACAAGGCCCTGCCTGCTCCTATCGAAAAGACCATCTCCAAGGCTAAGGGCCAGCCTCGGG
AACCTCAGGTTTACACACTGCCTCCATCTCGGGACGAGCTGACCAAGAATCAGGTGTCCCTGACCTGCCTCGTG
AAGGGCTTCTACCCTTCCGACATTGCCGTGGAATGGGAGAGCTATGGCACCGAGTGGTCCAGCTACAAGACAAC
CCCTCCTGTGCTGGACTCCGACGGCTCATTCTTCCTGTACTCCAAGCTGACCGTGACCAAGTCTGAGTGGCAGC
AGGGCTTCGTGTTCTCCTGCTCTGTGATGCACGAGGCCCTGCACAACCACTACACCCAGAAGTCCCTGTCTCTG
TCCCCTGGCAAATGAGTA
SEQ ID NO: 27 Amino acid sequence of hTREM2 as shown schematically in FIG. 3.

1. A protein capable of binding to human TREM2, comprising or consisting of an immunoglobulin (Ig) heavy chain variable region, wherein the amino acid sequence of the heavy chain variable region is that of SEQ ID NO: 2 or is an amino acid sequence having 1 or 2 amino acid residue substitutions compared to SEQ ID NO: 2, wherein said protein is capable of activating human TREM2-dependent pSYK signaling.

2. A protein, preferably according to claim 1, comprising an Ig light chain variable region and an Ig heavy chain variable region, wherein
the amino acid sequence of the light chain variable region is that of SEQ ID NO: 1 or is an amino acid sequence having 1 or 2 amino acid residue substitutions compared to SEQ ID NO: 1, and
the amino acid sequence of the heavy chain variable region is that of SEQ ID NO: 2 or is an amino acid sequence having 1 or 2 amino acid residue substitutions compared to SEQ ID NO: 2.

3. A protein capable of binding to human TREM2, comprising or consisting of a heavy chain variable region and/or comprising or consisting of a light chain variable region, wherein
the amino acid sequence of the heavy chain variable region is that of SEQ ID NO: 2 or is an amino acid sequence having 1 or 2 amino acid residue substitutions compared to SEQ ID NO: 2,
the amino acid sequence of the light chain variable region is that of SEQ ID NO: 1 or is an amino acid sequence having 1 or 2 amino acid residue substitutions compared to SEQ ID NO: 1;
wherein said protein is capable of activating human TREM2-dependent pSYK signaling.

4. The protein according to claim 1, comprising a light chain variable region and a heavy chain variable region, wherein
the light chain (LC) variable region comprises, preferably in CDR-L3, a segment of the amino acid sequence of SEQ ID NO: 5 or of an amino acid sequence having 1 amino acid residue substitution compared to the amino acid sequence of SEQ ID NO: 5; and
the heavy chain (HC) variable region comprises, preferably in CDR-H3, a segment of the amino acid sequence of SEQ ID NO: 8 or of an amino acid sequence having 1 amino acid residue substitution compared to the amino acid sequence of SEQ ID NO: 8.

5. The protein according to claim 2, wherein the light chain variable region comprises, preferably in CDRs L1 and L3, the following amino acid segments in N-terminal to C-terminal direction:
a segment (CDR-L1) of the amino acid sequence of SEQ ID NO: 3, and
a segment (CDR-L3) of the amino acid sequence of SEQ ID NO: 5 or an amino acid sequence having 1 amino acid residue substitution compared to the amino acid sequence of SEQ ID NO: 5; and
the heavy chain (HC) variable region comprises, preferably in CDRs H2 and H3, the following amino acid segments in N-terminal to C-terminal direction:
a segment (CDR-H2) of the amino acid sequence of SEQ ID NO: 7, and
a segment (CDR-H3) of the amino acid sequence of SEQ ID NO: 8 or of an amino acid sequence having 1 amino acid residue substitution compared to the amino acid sequence of SEQ ID NO: 8.

6. The protein according to claim 4, wherein the light chain (LC) variable region comprises, preferably in CDRs L1 to L3, the following amino acid segments in N-terminal to C-terminal direction:
a segment (CDR-L1) of the amino acid sequence of SEQ ID NO: 3,
a segment (CDR-L2) of the amino acid sequence of SEQ ID NO: 4, and
a segment (CDR-L3) of the amino acid sequence of SEQ ID NO: 5 or of an amino acid sequence having 1 amino acid residue substitution compared to the amino acid sequence of SEQ ID NO: 5; and
the heavy chain (HC) variable region comprises, preferably in CDRs H1 to H3, the following amino acid sequence segments in N-terminal to C-terminal direction:
a segment (CDR-H1) of the amino acid sequence of SEQ ID NO: 6,
a segment (CDR-H2) of the amino acid sequence of SEQ ID NO: 7, and
a segment (CDR-H3) of the amino acid sequence of SEQ ID NO: 8 or of an amino acid sequence having 1 amino acid residue substitution compared to the amino acid sequence of SEQ ID NO: 8.

7. The protein according to claim 1, wherein the protein is a single-chain antibody (scFv), an Fab fragment, an F(ab)₂ fragment, or an immunoglobulin (Ig); and/or
said protein is a fusion protein comprising a single-chain antibody (scFv), an Fab fragment, an F(ab)₂ fragment, or an immunoglobulin (Ig) as a first segment of the fusion protein and a second fusion protein segment.

8. The protein according to claim 1, comprising an antibody light chain (subunit) and an antibody heavy chain (subunit), said light chain comprising said light chain variable region and a light chain constant region, and
said heavy chain comprising said heavy chain variable region and at least one heavy chain constant region, preferably at least constant region CH1.

9. The protein according to claim 1, wherein said protein is an immunoglobulin selected from the group consisting of IgG, IgA, IgD, IgE, and IgM, or said protein is a fusion protein comprising said Ig and an additional fusion protein segment; and/or
wherein said protein is a fully human Ig generated in human immune cells.

10. The protein according to claim 2, comprising a light chain as follows:
(a) the amino acid sequence of said light chain is or comprises that of SEQ ID NO: 9, 10 or 11, or
(b) the amino acid sequence of said light chain is or comprises an amino acid sequence having 1 or 2 amino acid residue substitutions compared to the amino acid sequence of SEQ ID NO: 9, 10 or 11; and/or
comprising a heavy chain as follows:
(c) the amino acid sequence of said heavy chain is or comprises that of SEQ ID NO: 12, 13, 14, or 15, or
(d) the amino acid sequence of said heavy chain is or comprises an amino acid sequence having 1 or 2 amino acid residue substitutions compared to the amino acid sequence of SEQ ID NO: 12, 13, 14, or 15.

11. The protein according to claim 1, comprising a binding domain capable of binding to the human transferrin receptor 1 (hTfR1) for allowing crossing of said protein of the blood-brain-barrier, preferably said protein comprises a heavy chain having a modified C_{H}3 domain or comprises a C-terminal extension of the C_{H}3 domain that allows binding to the hTfR1.

12. The protein according to claim 1, wherein said protein is capable of binding to human TREM2 via its variable region, preferably to the stalk region of hTREM2; and/or said protein activates human TREM2-dependent human pSYK signaling; and/or is a hTREM2 agonist.

13. An antibody capable of binding to human TREM2, comprising an Ig light chain variable region as defined in claim 2 and an Ig heavy chain variable region as defined in claim 2, wherein said antibody is capable of activating human TREM2-dependent pSYK signaling.

14. The antibody according to claim 13, comprising:
a light chain wherein:
(a) the amino acid sequence of said light chain is or comprises that of SEQ ID NO: 9, 10 or 11, or
(b) the amino acid sequence of said light chain is or comprises an amino acid sequence having 1 or 2 amino acid residue substitutions compared to the amino acid sequence of SEQ ID NO: 9, 10 or 11; and
a heavy chain wherein:
(c) the amino acid sequence of said heavy chain is or comprises that of SEQ ID NO: 12, 13, 14, or 15, or
(d) the amino acid sequence of said heavy chain is or comprises an amino acid sequence having 1 or 2 amino acid residue substitutions compared to the amino acid sequence of SEQ ID NO: 12, 13, 14, or 15.

15. The antibody according to claim 13, comprising two light chains as follows:
(a) the amino acid sequence of said light chains is or comprises that of SEQ ID NO: 10 or 11, or
(b) the amino acid sequence of said light chains is or comprises an amino acid sequence having 1 or 2 amino acid residue substitutions compared to the amino acid sequence of SEQ ID NO: 10 or 11; and
two heavy chains as follows:
(c) the amino acid sequence of said heavy chains is or comprises that of SEQ ID NO: 12, 13, 14, or 15, or
(d) the amino acid sequence of said heavy chains is or comprises an amino acid sequence having 1 or 2 amino acid residue substitutions compared to the amino acid sequence of SEQ ID NO: 12, 13, 14, or 15.

16. Pharmaceutical composition comprising the protein or antibody according to claim 1 and a pharmaceutically acceptable carrier.

17. (canceled)

18. (canceled)

19. (canceled)

20. (canceled)

21. (canceled)

22. Nucleic acid molecule encoding a protein, antibody, light chain and/or heavy chain as defined in claim 1, or comprising or consisting of a nucleotide sequence of SEQ ID NO: 25 or 26.

23. Eukaryotic cell comprising a protein according to claim 1.

24. A method of treating or preventing of a neurodegenerative disease, such as Alzheimer's disease, comprising administering a protein or antibody as defined in claim 1 to a mammal in need thereof.

25. The method according to claim 24, comprising administering the protein or antibody to a human patient that is at an early stage of said disease.

26. The method according to claim 24, comprising administering the protein or antibody to a human patient having a stage of the disease:
at a score of 23 or lower, preferably at a score of from 10 to 23, more preferably at a score of from 19 to 23, cognitive impairment of the patient in the mini-mental state examination (MMSE) test, or
at a score of 0.5 or higher and 2 or lower cognitive impairment of the patient on the Clinical Dementia Rating Scale (CDR Global Score).

27. The method according to claim 24, comprising parenteral administration of said protein or antibody to a mammal, preferably intravenous, subcutaneous or intraperitoneal administration.

28. Eukaryotic cell comprising a nucleic acid molecule according to claim 22.
